(19) United States
(12) Patent Application Publication
(10) Pub. No.: US 2007/0037143 A1
(43) Pub. Date: Feb. 15, 2007

(54) Method for screening for protease modulators

(75) Inventors: Marco Jost, Bonn (DE); Jens Lamerz, Hannover (DE); Petra Budde, Hannover (DE); Horst Rose, Hannover (DE); Norbert Lamping, Hannover (DE); Rainer Voegeli, Wielenbach (DE); Harald Tammen, Hannover (DE)

Correspondence Address: EDWARDS & ANGELL, LLP, P.O. BOX 55874, BOSTON, MA, 02205, US

(73) Assignee: DIGILAB BioVisioN GmbH, Hannover (DE)

(21) Appl. No.: 11/445,830

(22) Filed: Jun. 1, 2006

Related U.S. Application Data
(60) Provisional application No. 60/687,271, filed on Jun. 2, 2005.

(51) Int. Cl. C12Q 1/70; C12Q 1/37

(52) U.S. Cl. 435/5; 435/23

(57) ABSTRACT

The present invention is in the field of pharmaceutical research tools. Specifically, the present invention provides methods for screening compounds or compositions with respect to their ability and/or specificity to modulate the activity of proteases.

### CROSS-REFERENCE TO RELATED APPLICATIONS

This application claims priority to U.S. Provisional Patent Application No. 60/687,271 filed Jun. 2, 2005, which is hereby expressly incorporated herein in its entirety by this reference.

### FIELD OF THE INVENTION

The present invention is in the field of pharmaceutical research tools. Specifically, the present invention provides methods for screening compounds or compositions with respect to their ability and/or specificity to modulate the activity of proteases.

### BACKGROUND OF THE INVENTION

It is known that proteases play important roles in normal cell functions and in diseases and are targets for certain drugs in the treatment of, for example, cancers, parasitic, fungal and viral infections, and inflammatory, immunological, respiratory, cardiovascular and neurodegenerative disorders (Leung et al., J. Med. Chem. 43: 305-41, 2000). It is estimated that the human genome encoded between 1000 and 2000 proteases, among which many will be potential targets for drug discovery projects (Southan, Drug Discov. Today 6: 681-8, 2001; Southan, FEBS Lett. 498: 214-8, 2001). Protease regulators, especially protease inhibitors, are strongly desired for their potential therapeutic applications.

Several methods are available in the field for screening protease inhibitors. Commonly, the first step leading to the design of the appropriate protease inhibitors for therapeutics is the characterization of substances in in vitro assay systems with respect to their ability to inhibit the activities of proteolytic enzymes. Most of these methods utilize a peptide or chemical substrate and are based on that the protease cleavage would release a covalently bound reporter group of a chromogenic, fluorogenic, or immunological activity, with each having advantages and disadvantages in terms of sensitivity, specificity, cost, assay time, equipment needs, and background level.

Accordingly, there is a need for an effective and sensitive screening method for the detection of candidate substances having the ability to modulate the activity of proteolytic enzymes such as protease inhibitors and activators.

### SUMMARY OF THE INVENTION

The present invention generally is directed to methods for identification of substances with functional activity towards proteases and fulfills the need for effective screening for protease modulators, in particular protease inhibitors.

The methods according to the invention allow for the screening for protease modulators in an environment where the proteases used in testing are naturally present, in particular within a living organism. Furthermore, the methods of the invention do not require the purification of proteases to be used in the screening and therefore, do not have the risk of changing the activity and/or specificity of the proteases, in particular inactivating one or more proteases, due to purification procedures. In addition, the methods of the invention do not require the addition of protease inhibitors which are frequently harmful to the user. The methods of the invention offer the possibility of testing one or more compounds or compositions, proteases and/or substrates for proteases within a single test system and using naturally occurring substrates without purification of and/or artificially introducing labels into said substrates which can result in a change of the specificity for and/or activity towards said substrates of one or more proteases compared to the naturally occurring substrates. Thus, the methods of the invention allow for the use of substrates for one or more proteases which reflect the naturally occurring substrate usage of said one or more proteases.

Furthermore, those embodiments of the methods of the invention which are partially or completely performed within a living organism allow for the early determination whether a substance being tested is harmful, in particular toxic, carcinogenic and/or lethal, for the living organism which might be a reason for not testing said substance further.

The methods of the present invention may be used to screen for modulators of any proteases of interest. In addition to screening for modulators of naturally occurring proteases (including naturally occurring variants of wild type proteases), the present invention may also be used to screen for modulators of proteases that are artificially mutagenized but remain enzymatically active. Preferred proteases include those that have been implicated in human diseases. In addition any compound or composition suspected to modulate the activity of proteases may be tested using the method of the present invention.

Specifically, the invention relates to a method of determining the ability of a compound or composition to modulate the activity and/or the specificity of at least one protease comprising the steps of a) contacting said compound or composition with at least one protease in the presence of a plurality of peptides and/or proteins, and optionally in the presence of a protease inhibitor or protease activator, and b) determining qualitatively and/or quantitatively the peptides and/or proteins resulting from step (a).

In a preferred embodiment, step (b) is performed in a time dependent manner. In certain embodiments of this method of the invention, step (b) is performed at a first time and at least a second time.

In certain embodiments, the method of the invention further comprises the step of comparing the results obtained in step (b) with a reference sample, wherein said reference sample preferably contains a known modulator of the activity of said at least one protease.

In preferred embodiments, the method of the invention further comprises the step of: c) comparing the results obtained in step (b) with the results obtained from (i) determining qualitatively and/or quantitatively the peptides and/or proteins prior to step (a) and/or (ii) determining qualitatively and/or quantitatively the peptides and/or proteins in a corresponding sample which does not contain said compound or composition and/or (iii) determining qualitatively and/or quantitatively the peptides and/or proteins in a corresponding sample which contains a different concentration of said compound or composition and/or (iv) determining qualitatively and/or quantitatively the peptides and/or proteins in a corresponding sample which contains a different compound or composition and/or (v) determining qualitatively and/or quantitatively the peptides and/or proteins in a corresponding sample which has been contacted with said compound or composition for a different period of time.

Preferably, step (a) takes place within in vitro cultured cells, within the culture medium of in vitro cultured cells or within a living organism. In those embodiments where step (a) takes place within a living organism the method of the invention may further comprise the step of: c) comparing the results obtained in step (b) with the results obtained from at least one further living organism. In preferred embodiments, said at least one further living organism (i) has not been exposed to said compound or composition or (ii) has been exposed to a different compound or composition or (iii) has been exposed to a different concentration of said compound or composition and/or (iv) has been exposed to said compound or composition for a different period of time.

In certain aspects of the method of the invention, said first living organism and said at least one further living organism have been subjected or are subjected to different environmental conditions, and/or are of different age, sex, weight and/or health condition. In further aspects of the method of the invention said living organism and/or said at least one further living organism is afflicted with a disease wherein in certain embodiments said disease is selected form the group consisting of hypertension, HIV/AIDS, haemophilia, thrombosis, cancer, inflammatory diseases, type II diabetes, Alzheimer's disease, osteoporosis, and hepatitis C infection (HCV).

In certain embodiments of the method of the invention a compound or composition to be tested may be administered to a living organism having a compromised health condition, in particular a living organism being afflicted with a disease, whereby said compromised health condition, in particular said disease quantitatively and/or qualitatively alters the peptide and/or protein pattern within at least parts, e.g. certain body fluids, of said living organism, compared to a healthy living organism, and it may be determined by the methods of the invention whether the administration of said compound or composition partially or fully restores a peptide and/or protein pattern of a healthy living organism, i.e. a living organism not having said compromised health condition, in particular a living organism not being afflicted with said disease, within said living organism having said compromised health condition, in particular said living organism being afflicted with said disease. In these embodiments the partial or full peptide and/or protein pattern of the living organism having a compromised health condition or being afflicted with a disease after exposure to the compound or composition can be compared with the partial or full peptide pattern of a living organism not having said compromised health condition or not being afflicted with said disease and/or the partial or full peptide and/or protein pattern of a living organism having said compromised health condition or being afflicted with said disease which (i) has not been exposed to said compound or composition or (ii) has been exposed to a different compound or composition or (iii) has been exposed to a different concentration of said compound or composition and/or (iv) has been exposed to said compound or composition for a different period of time.

The methods of the invention may also be used for screening for harmful, in particular toxic and/or carcinogenic substances by exposing a living organism to a substance, i.e. compound or composition, and determining whether a partial or full peptide and/or protein pattern of said living organism adopts a pathogenic status, in particular partially or fully resembles the peptide and/or protein pattern of a living organism having a compromised health condition, e.g. a living organism being afflicted with a disease. In these embodiments a partial or full peptide and/or protein pattern of the living organism after exposure to the compound or composition can be compared with a partial or full peptide pattern of a living organism having a compromised health condition, e.g. a living organism being afflicted with a disease, wherein the expression “compromised health condition” relates to any deviation in the state of health of a living organism when compared to a healthy living organism.

Said first living organism and said at least one further living organism may be of the same or different species, wherein said living organisms of the same species may have different genetic backgrounds. Furthermore, said living organism and/or said at least one further living organism may be a unicellular organism such as bacteria or yeast or multicellular organism, preferably a human being or nonhuman primate or another animal, in particular a mammal such as a cow, horse, pig, sheep, goat, dog, cat or a rodent such as a mouse and rat. In a particularly preferred embodiment, the living organism is a human being.

In those embodiments of the method of the invention wherein step (a) takes place within a living organism said compound or composition and/or said at least one protease may be delivered, preferably administered such as by injection or oral, nasal, topical administration, to said living organism and/or said at least one further living organism.

Preferably, the method of the invention further comprises the step of comparing the results obtained in step (b) with clinical or other meta data.

In further preferred embodiments of the method of the invention, said ability of a compound or composition to modulate the activity of at least one protease is the ability to decrease or increase the activity of said at least one protease.

Preferably, in the method of the invention said at least one protease comprises at least one protease selected from the group consisting of aspartic peptidases, cysteine peptidases, glutamic peptidases, metallo peptidases, serine peptidases, threonine peptidases, amino peptidases, carboxypeptidases, endopeptidases, exopeptidases, in particular at least one protease selected from the group consisting of angiotensin converting enzyme (ACE), ACE2, renin, neutral endopeptidase (NEP), HIV-1 protease, HIV-2 protease, factor Xa, factor VIIa, factor XIIIa, interleukin-1 converting enzyme (ICE), dipeptidyl peptidase 4 (DPP4), alpha-, beta-, gamma-secretase, caspase, cathepsin K, Matrix metalloproteinases (MMPs), proteosome protease, NS3 protease.

The step of determining qualitatively and/or quantitatively the peptides and/or proteins in the method of the invention may comprise the step of determining the relative or absolute quantity of said peptides and/or proteins and said quantitative and/or qualitative determination of said peptides and/or proteins may be performed using mass spectrometric methods, preferably in combination with separation methods for said peptides and/or proteins. In particular embodiments, said separation methods are performed prior to, simultaneous with and/or subsequent to said mass spectrometric methods and may be selected from the group consisting of gel electrophoresis, liquid chromatography, gas chromatography, capillary chromatography, thin layer chromatography, mass spectrometry, precipitation techniques, liquid phase extraction techniques, filtration and other molecular size discriminating techniques.

In the method of the invention, the mass spectrometric methods may be selected from the group consisting of MALDI mass spectrometry, ESI mass spectrometry, SELDI mass spectrometry, FAB mass spectrometry and MS/MS mass spectrometry.

In particular preferred embodiments of the method of the invention said plurality of peptides and/or proteins is comprised in a biological sample which may be selected, for example, from the group consisting of blood, serum, plasma, urine, liquor, bronchial aspirate, sputum, tissue extracts, cell extracts, cell culture medium.

In certain embodiments, the methods of the invention further comprise obtaining the protease and/or the protease inhibitor or the protease activator. As used herein, the term “obtaining” as in obtaining a substance includes synthesizing, purchasing or otherwise acquiring the substance.

The invention also provides kits for determining the ability and/or the specificity of a compound or composition to modulate the activity of a protease. In one embodiment, the kit comprises at least one protease and instructions for using the protease to determine the ability and/or the specificity of a compound or composition to modulate the activity of the protease in accordance with the methods of the invention. In another embodiment, the kit further comprises a protease inhibitor or a protease activator.

In yet another embodiment, the kit comprises a protease inhibitor or a protease activator and instructions for using the protease inhibitor or protease activator to determine the ability and/or the specificity of a compound or composition to modulate the activity of a protease in accordance with the methods of the invention.

### BRIEF DESCRIPTION OF THE DRAWINGS

FIG. 1:

50 to 800 pmol of neurotensin were introduced into plasma samples, subsequently separated by reversed phase chromatography (RP-chromatography) and those fractions containing neurotensin were analyzed by mass spectrometry as described in the examples. There is a linear correlation between the MALDI mass spectrometry signal intensity (y-axis) of the neurotensin peptide and the amount of the neurotensin peptide added to the plasma sample (x-axis), indicating that MALDI mass spectrometry is suitable to quantitatively determine peptides in complex samples such as plasma.

FIG. 2:

Wister rats were either injected with various amounts of the irreversible DPP4 inhibitor AB192 (8 animals per dosage), control inhibitor (Contr.In.) (8 animals per dosage) or vehicle (4 animals). Blood samples were taken immediately prior and 1 h after injection. Plasma was prepared and used for a DPP4 activity assay. The figure shows the DPP4 activity measured in U/L (Units/litre; y-axis) versus the concentration of inhibitor injected (mg/kg; x-axis) for AB192 (panel A) and Contr.In. (panel B). This indicates that both inhibitors inhibit DPP4 activity in vivo in a dose-dependent manner.

FIG. 3:

FIG. 3 shows the mass spectrometric signal intensity relative to the DPP4 activity determined in plasma samples of individual rats. Rats were treated with the control inhibitor (Contr.In.). Panel A shows the results of the Peptide/Protein no. I identified in fraction 23 (Frac. 23) and having a mass of 1737 (m/z 1737) (Table 1). The correlation shows that the increase in the mass spectrometric signal of Peptide/Protein no. 1 correlates positively and linear with the DPP4 activity measured in the same samples. Panel B shows the results of the Peptide/Protein no. 2 identified in fraction 42 and having a mass of 2323 (Table 1). The correlation shows that the increase in the mass spectrometric signal of Peptide/Protein no. 2 correlates positively and linear with the amount of the DPP4 inhibitor AB192 or the control inhibitor given to the experimental rat.

FIG. 4:

FIG. 4 shows data similar to those presented in FIG. 3 for different peptides/proteins. Panel A shows the results of the Peptide/Protein no. 3 identified in fraction 30 and having a mass of 1369 (Table 1). The correlation shows that the increase in the mass spectrometric signal of Peptide/Protein no. 3 correlates positively and linear with the amount of control inhibitor (Contr.In.) given to the rat. Panel B shows the results of the Peptide/Protein no. 45 identified in fraction 21 and having a mass of 2579 (Table 1). The correlation shows that the increase in the mass spectrometric signal of Peptide/Protein no. 45 correlates negatively with the DPP4 activity measured in the same samples of rats treated with the control inhibitor.

FIG. 5:

FIG. 5 shows panels A to C each representing combinations of two peptides or proteins from Table 1 (Peptide/Protein no. 56 and 16 of Table 1 form panel no. 62 of Table 2). A combination of these peptides or proteins to form a peptide/protein panel improves the correlation value as compared to the correlation values of the individual peptides/proteins. When used individually Peptide/Protein no. 56 results in a correlation coefficient r of −0.58 (panel A) and Peptide/Protein no. 16 results in a correlation coefficient r of 0.55 (panel B). When combining both peptides/proteins to form a panel, the correlation coefficient r is improved to 0.73 (panel C).

FIG. 6:

FIG. 6 shows the results of the determination of the clotting activity (Example 9) present in plasma of rats treated with clotting activator in the absence (left 3 columns) and in the presence (right 3 columns) of a clotting inhibitor. The clotting activity was measured using a commercially available thrombin-antithrombin (TAT) complex ELISA. Plasma samples from rats used in the experiments were obtained directly prior to infusion of the clotting activator (time=0 min) and 30 min and 60 min after infusion of the clotting activator. FIG. 6 shows that there was only a low baseline level of clotting activity at t=0, whereas the clotting activity was clearly increased at t=30 and t=60 min. In the presence of the clotting inhibitor the clotting activity was clearly reduced by about 50%.

FIG. 7:

FIGS. 7A and 7B show details of single fractions of 14 individual peptide/protein maps obtained from plasma samples of 14 individual rats in 2 independent experiments. The rats were infused either 0.9% saline (vehicle control group; symbol in the figure: - - -), 0.9% saline together with a clotting activator (clotting activator control group; symbol in the figure: +A) or clotting inhibitor and subsequently clotting activator (clotting inhibitor group; symbol in the figure: +I+A). The axis at the top indicates the mass/charge (m/z) value of the peptides shown in the section of the peptide maps.

FIG. 8:

FIG. 8 shows the schematic work flow from performing reversed phase chromatography (panel A), measuring of individual fractions by mass spectrometry and converting the spectra in a “gel-like” view (panel B), combining the 96 “gel-like” views of mass spectra of one sample to a peptide/protein pattern (so called peptide display) of that sample (panel C), electronically subtracting the signal intensities of peptide/protein patterns obtained from different samples (for example, serum versus plasma), from each other resulting in a differential peptide display (panel D), to determining the sequence of selected peptides by mass spectrometry (panel E).

FIG. 9:

FIG. 9 shows the result of electronically subtracting the signal intensities of plasma samples from serum samples. The resulting differential peptide display shows all peptides and/or proteins which differ in their amount present in serum and plasma.

FIG. 10:

FIG. 10 shows peptide displays of EDTA plasma, citrate plasma, heparin plasma and serum.

FIG. 11:

FIG. 11 shows the result of electronically subtracting the signal intensities of serum samples clotted for 15 min from serum samples clotted for 8 hours. The resulting differential peptide display shows all peptides and/or proteins which differ in their amount present in the two samples in a clotting time-dependant manner.

### DETAILED DESCRIPTION OF THE INVENTION

Ability and/or Specificity to Modulate the Activity of at Least One Protease

The term “ability of a compound or composition to modulate the activity of at least one protease” designates the potency of a substance, i.e. compound and/or composition, if in contact with at least one protease to result in a change in the activity and/or specificity of said at least one protease compared with the activity and/or specificity of said at least one protease before or without contact with said substance. This includes the situation where the substance increases or decreases the activity of said at least one protease. Furthermore, it includes the situation where the substance results in an altered specificity of said at least one protease for the substrate and/or other binding substances such as co-factors. Also included are substances altering the expression, half-life, etc. of said at least one protease.

In general, if a substance is tested for its ability to modulate the activity of at least one protease, i.e. the ability to modulate the activity of at least one protease is determined, it will be unknown before such testing whether the substance tested has such ability or not. However, in other embodiments there may be indications or information that a substance to be tested has such ability or not. This includes, for example, situations where it is already known that a substance increases or decreases the activity of at least one protease and it is tested by the methods of the invention whether said substance also has the ability to alter the specificity of at least one protease. It also includes situations where a substance has already been tested for its ability to modulate the activity of at least one protease and the substance is tested by the methods of the invention to confirm the earlier results or to test the substance in a different system and/or environment or to compare the substance with another substance. In particular it is possible to compare the substance with another substance, preferably a known substance, of which substance the activity, specificity and possible adverse side effects are already known.

The wording “determining the specificity of a compound or composition to modulate the activity of one or more proteases” includes situations where it was determined previously, for example by the methods of the present invention, whether a substance has the ability to modulate the activity of at least one protease and said substance is tested by the methods of the present invention with respect to said ability in a different environment, for example with different proteases, substrates and/or in a different background such as in the presence or absence of other compound such as co-factors, to test, for example, whether the ability of a compound or composition to modulate the activity of at least one protease is specific for particular proteases, groups or families of proteases, particular substrates etc.

In those embodiments of the methods of the invention where two or more proteases are present in a sample, the expression “modulate the activity of least one protease” includes situations where the activity of none or one or more of the proteases or a particular group or family of the proteases present in the sample is modulated, wherein if the activity of two or more of the proteases present in the sample is modulated this modulation can be of different nature, e.g. the activity of one or more of said proteases may be increased and the activity of one or more of said proteases may be decreased by a compound or composition tested.

Determining Qualitatively and/or Quantitatively Peptides and/or Proteins

According to the invention, the activity of at least one protease, which term relates to the degree of activity, i.e. high, low, or no activity, as well as specificity of at least one protease is measured by determining the amount of peptides and/or proteins present in the tested sample after said at least one protease has been contacted with a compound or composition to be tested for its ability and/or specificity to modulate the activity of said at least one protease. “Determining the amount of peptides and/or proteins” in this regard preferably relates to the absolute and/or relative quantification of said peptides and/or proteins wherein said peptides and/or proteins may be substrates for said at least one protease or products formed by the activity of said at least one protease or neither substrates for nor products formed by the activity of said at least one protease or combinations thereof. The higher the activity of the proteases in a sample, the more substrate will be transformed into product by said proteases. Accordingly, the amount of substrate determined in a sample will decrease with increasing activity of protease in the sample and the amount of product will increase in the sample. Thus, a small amount of substrate and/or large amount of product determined in a sample is indicative for a high activity of protease in the sample which in turn may be indicate for the ability of a tested substance to activate at least one protease. A large amount of substrate and/or small amount of product determined in a sample is indicative for a low activity of protease in the sample which in turn may be indicate for the ability of a tested substance to inhibit at least one protease.

The expression “determining qualitatively and/or quantitatively peptides and/or proteins” according to the invention also includes situations wherein no peptide and/or protein which is a product formed by the activity of said at least one protease is detected or the amount of said peptide and/or protein formed is below the detection limit which situations may be indicative for the fact that protease present in the sample is substantially or completely inactive or the activity of the protease is substantially or completely inhibited.

Furthermore, the expression “determining qualitatively and/or quantitatively peptides and/or proteins” also includes situations wherein a peptide and/or protein which is a substrate for said at least one protease is not detectable either because it is not present in the sample or because its concentration is below the detection limit of the test system used which situations may be indicative for the fact that protease present in the sample is highly active which in turn may be indicative for the fact that the tested substance activates the at least one protease.

On the basis of the amount of peptides and/or proteins determined in a sample it is possible in the methods of the invention to draw conclusions about the activity and/or specificity of at least one protease in the sample and in turn about the ability and/or specificity of a tested substance to modulate the activity of said at least one protease. In this respect, the activity of a protease may be calculated as a relative or an absolute activity, depending on the experimental setup.

Expressions such as “substrate for said at least one protease”, “product formed by the activity of said at least one protease”, “modulate the activity of said at least one protease” include but are not limited to situations wherein a substance is a substrate for all proteases present in a sample, or a product is formed by the activity of all proteases present in a sample or the activity of all proteases present in a sample is modulated. However, such expressions, for example, also include situations where a substance is a substrate for only a fraction, i.e. one or more, group or one or more families of proteases present in a sample, a product is formed by the activity of only a fraction, i.e. one or more, group or one or more families of proteases present in a sample or the activity of only a fraction, i.e. one or more, group or one or more families of proteases present in a sample is modulated.

Preferably, the amount of peptides and/or proteins in a test sample to which a candidate substance has been added is compared with a reference sample. This allows to draw conclusions about whether the addition of a candidate substance has changed the activity and/or specificity of at least one protease in the test sample which is expressed in a different qualitative and/or quantitative pattern of the peptides and/or proteins in the test sample compared to the reference sample.

An experimental setup to determine the relative concentration or activity of at least one protease may be conducted, for example, by measuring the amount or concentration of a substrate of said at least one protease or a product formed by the activity of said at least one protease in a test sample. This amount or concentration of a substrate of said protease or a product formed by the activity of said protease may be compared with a reference value obtained from a reference sample not containing the test substance. Thus, it is possible to e.g. determine the specificity and/or activity of said protease in the test sample. The amount of substrate or product can be reflected by a signal intensity in a test system, for example a mass spectrometric signal intensity, an absorption value, fluorescence value, radioactive counts, luminescence value, etc. of an ELISA (enzyme-linked immuno-sorbent assay), a RIA (radio immuno assay), a protein- or DNA-chip assay, a FACS (fluorescence activated cell sorting) analysis, an immune precipitation, a quantitative PCR (polymerase chain reaction) or RT-PCR (reverse transcriptase-PCR) reaction, or densitometric signal intensity of an exposed film (for example from a Western blot, a Northern blot, or an in situ hybridization), etc.

An experimental setup to determine the activity of a protease may be conducted, for example, by quantitatively determining the concentration of a substrate of said protease or a product formed by the activity of said protease, for example by means of an ELISA or by quantitative mass spectrometric measurement of the mass signal of a peptide which is a substrate or product of said protease. Furthermore, by empirically determining with various known concentrations or various known quantities of activities of said protease the reduction in substrate concentration or increase in product concentration over time, it is possible to establish a standard curve for determining the absolute activity of said protease in a test sample.

However, as described herein, it is not necessary in the methods of the invention to identify and/or characterize which peptides and/or proteins in a sample are substrates of or products formed by the activity of proteases. It is also not necessary to know the identity (the sequence) of any, some or all of the plurality of peptides and/or proteins accompanied by said at least one protease contacted with a compound or composition. In particular embodiments of the methods of the invention, if the amount of a peptide and/or protein decreases after contact with at least one protease, this will be indicative for the fact that said peptide and/or protein is a substrate for said at least one protease. Vice versa, in particular embodiments of the methods of the invention, if the amount of a peptide and/or protein increases after contact with at least one protease, this will be indicative for the fact that said peptide and/or protein is a product formed by the activity of said at least one protease.

Contacting a Compound or Composition with at Least One Protease

According to the invention, the term “protease” relates to enzymes that catalyze the splitting of proteins into smaller peptides and/or amino acids by a process known as proteolysis [syn: peptidase, proteinase, proteoyltic enzyme].

Said term comprises proteases such asaspartic peptidases, cysteine peptidases, glutamic peptidases, metallo peptidases, serine peptidases, threonine peptidases, amino peptidases, carboxypeptidases, endopeptidases, exopeptidases wherein specific examples are angiotensin converting enzyme (ACE), ACE2, renin, neutral endopeptidase (NEP) (involved in hypertension), HIV-1 protease, HIV-2 protease, factor Xa, factor VIIa, factor XIIIa (involved in thrombosis), interleukin-1 converting enzyme (ICE) (involved in inflammatory diseases), dipeptidyl peptidase 4 (DPP4) (involved in type II diabetes), alpha-, beta-, gamma-secretase, caspase (involved in Alzheimer's disease), cathepsin K (involved in osteoporosis), Matrix metalloproteinases (MMPs), proteosome protease (involved in cancer), NS3 protease (involved in HCV).

However, in the methods of the invention the proteases used may not be characterized, i.e. it is not necessary to know their chemical composition, structure, specificity etc.

The expression “contacting a compound or composition with at least one protease” refers to the situation that at least one protease and a substance to be tested, i.e. compound or composition, are brought and come into contact. The substrate(s) for said at least one protease can be present in the sample in situ or can be added to the sample. However, the present invention also includes situations wherein one or more substrates are present in a sample in situ and, in addition, one or more of the same or different substrates are added to the sample.

The expression “at least one protease” means one or more, preferably 2 or more, more preferably 3 or more, 4 or more, 5 or more, 6 or more, 8 or more, 10 or more, 15 or more, even more preferably 20 or more, 30 or more, 40 or more, 50 or more, 60 or more, 70 or more, 80 or more, 90 or more, or 100 or more different proteases wherein the term “different proteases” means that the proteases are not identical and preferably differ in their chemical composition such as amino acid sequence, substrate specificity, activity, requirement of other factors, temperature and pH preferences and/or belong to different families or groups of proteases such as aspartic peptidases, cysteine peptidases, glutamic peptidases, metallo peptidases, serine peptidases, threonine peptidases, amino peptidases, carboxypeptidases, endopeptidases, exopeptidases. The upper limit of said “at least one protease” is not particularly restricted as long as the number of the peptides and/or proteins produced by the activity of said at least one protease does not exceed the resolution limit of the assay system. An upper limit may be for example not more than 20, 40, 60, 80, 100, 150, 200, 250, or 300 proteases depending on the assay system used in the methods of the invention. In certain embodiments of the methods of the invention, only a part or fraction of the peptides and/or proteins resulting from proteolytic cleavage are determined which may allow a higher upper limit of said “at least one protease”. In these embodiments, the peptides and/or proteins to be determined may be subjected to separation means such as chromatographic separations before being determined.

In preferred embodiments of the methods of the present invention said at least one protease is present within or derived from a biological sample or fraction of a biological sample. However, this does not exclude the possibility of adding further proteases derived from the same or other sources which then form said “at least one protease” together with proteases present in said biological sample or fraction of a biological sample.

In those embodiments of the present invention wherein said at least one protease is present within a biological sample the step of contacting a substance, i.e. compound or composition, with said at least one protease in the presence of a plurality of peptides and/or proteins may take place in vivo, i.e. within a living organism. Furthermore, in these embodiments, the substance to be tested is preferably delivered, in particular by administration such as injection or oral administration, to said living organism.

In particular embodiments of the methods of the present invention the activity of particular proteases and/or groups of proteases present within said at least one protease is decreased and/or increased preferably by the addition of specific known protease inhibitors or activators.

Substrates for and Products Formed by the Activity of Proteases

According to the invention, the ability and/or specificity of a substance, i.e. compound or composition, to modulate the activity of at least one protease is measured by determining the activity and/or specificity of at least one protease that has been contacted with said substance in the presence of a plurality of peptides and/or proteins wherein one or more of said plurality of peptides and/or proteins can serve as substrates for said at least one protease. For the methods of the invention, however, it is not necessary to identify which peptides and/or proteins in said plurality of peptides and/or proteins can serve as substrates for said at least one protease and/or determine their composition. The present invention is based on the observation that by determining peptide and/or protein patterns of samples comprising a plurality of peptides and/or proteins it is possible to identify changes in the activity and/or specificity of one or more proteases present within the sample. Thus, the methods of the present invention rely on the determination of differences and/or changes in the partial or entire peptide and/or protein pattern of a sample, in particular a biological sample, rather than on the identification of the composition of the peptides and/or proteins, in particular substrates and products of proteoyltic activity, within a sample. However, the methods of the present invention do not exclude embodiments wherein the composition of peptides and/or proteins is identified.

In this respect, the term “identification of the composition of peptides and/or proteins” relates to the full or partial identification of the chemical structure of one or more of said peptides and/or proteins and in particular, the full or partial identification of the sequence of one or more of said peptides and/or proteins.

The expression “plurality of peptides and/or proteins” means two or more, preferably 4 or more, more preferably 6 or more, 8 or more, 10 or more, 15 or more, even more preferably 20 or more, 30 or more, 40 or more, 50 or more, 60 or more, 70 or more, 80 or more, 90 or more, 100 or more, 120 or more, 140 or more, 160 or more, 180 or more, 200 or more, 250 or more, 300 or more, 400 or more, 500 or more different peptides and/or proteins. The upper limit of said “plurality of peptides and/or proteins” is not particularly restricted as long as the number of said peptides and/or proteins does not exceed the resolution limit of the assay system. An upper limit may be for example not more than 20, 40, 60, 80, 100, 150, 200, 250, 300, 400, 450, 500, 1000, 2000, 5000 or 10000 peptides and/or proteins depending on the assay system used in the methods of the invention. In certain embodiments of the methods of the invention, only a part or fraction of the peptides and/or proteins resulting from proteolytic cleavage are determined which may allow a higher upper limit of said “plurality of peptides and/or proteins”. In these embodiments the peptides and/or proteins to be determined may be subjected to separation means such as chromatographic separations before being determined.

In preferred embodiments of the methods of the present invention said plurality of peptides and/or proteins is present within or derived from a biological sample or fraction of a biological sample. However, this does not exclude the possibility of adding further peptides and/or proteins derived from the same or other sources which then form said “plurality of peptides and/or proteins” together with peptides and/or proteins present in said biological sample or fraction of a biological sample.

In those embodiments of the present invention wherein said plurality of peptides and/or proteins is present within a biological sample the step of contacting a substance, i.e. compound or composition, with at least one protease in the presence of said plurality of peptides and/or proteins may take place in vivo, i.e. within a living organism. Furthermore, in these embodiments, the substance to be tested is preferably delivered to said living organism.

According to the invention, a substrate for a protease comprises at least one bond susceptible to cleavage by said protease. Preferably, a substrate for a protease is a peptide or protein. However, such substrate may also comprise non-peptide or non-protein portions. Accordingly, the product formed by the activity of said protease is a peptide or protein which may also include non-peptide or non-protein portions.

Peptides or proteins described herein may be of any length. A peptide or protein described herein preferably comprises a sequence of at least 4 or 6, in particular at least 8, at least 10, at least 12, at least 15, at least 20, at least 30 or at least 50 consecutive amino acids.

Furthermore, the peptides or proteins described herein may be variants. A “variant” as used herein, comprises a peptide or protein that differs from a peptide or protein from which it is derived, in particular a naturally occurring peptide or protein, in one or more substitutions, deletions, additions and/or insertions of amino acids. Such variants may be those that exist naturally, or those in which one or more substitutions, deletions, additions and/or insertions have been introduced.

Peptide or protein variants according to the invention include those exhibiting at least about 70%, 75%, 80%, 85%, 90%, 91%, 92%, 93%, 94%, 95%, 96%, 97%, 98%, or 99% or more identity to the peptide or protein sequences from which they are derived.

The peptides or proteins described herein may also comprise any natural and non-natural modifications, in particular chemical or physical modifications. The term “natural modification” relates to any modification found in peptides or proteins in nature such as posttranslational modifications, modifications due to exposure to light, oxygen, exposure to acid or alkali solutions, etc.

Preferably the peptides or proteins described herein are modified to facilitate their detection, alter their stability during storage or usage (for example by using peptidomimetic structures), alter their toxicity or bioavailability or biological half-life, etc. (for example by pegylation). Preferred modifications are those which facilitate the detection of peptides or proteins such as fluorescent labels, chromophore labels, luminescent labels, radioisotopic labels, isotopic labels, isobaric labels, enzyme labels, particle labels, small organic molecules such as biotin, ligands of receptors or binding molecules such as cell adhesion proteins or lectins, label-sequences comprising nucleic acids and/or amino acids which can be detected by use of binding agents, etc. Label sequences can be bound by binding agents such as antibodies, receptors, PCR-primers, Northern blot probes, etc. Label sequences can be amino acid sequences or nucleic acid sequences or combinations thereof or molecules mimicking the structure of amino acid or nucleic acid sequences (mimetics).

Labels for Peptides and Proteins

The invention envisions the use of peptides and proteins, which comprise at least one label. The term “label”, as used herein, refers to any moiety that functions to: (i) provide a detectable signal; (ii) interact with a second label to modify the detectable signal provided by the first or second label, e.g. FRET (Fluorescence Resonance Energy Transfer); (iii) affect mobility, e.g. electrophoretic mobility, by charge, hydrophobicity, shape, or other physical parameters, or (iv) provide a capture moiety, e.g., affinity, antibody/antigen, or ionic complexation.

Suitable as label are all kinds of structures, such as isotopic labels, isobar labels, fluorescent labels, radioactive labels, luminescent labels, enzyme labels, toxin labels, dye labels, metal particles as labels, magnetic particles as labels, polymer particles as labels, biotin or other organic molecules as labels, etc.

For example, isotopes with molecular weights different to their natural molecular weight such as oxygen-18, nitrogen-15, deuterium, tritium, carbon-14, sulphur-35, phosphorus-32 or phosphorus-33 etc. can be used to prepare isotope-labelled peptides or proteins. The isotopes can be incorporated directly into the peptides or proteins by using isotope-labelled amino acids for synthesis of the peptides or proteins. Furthermore, these isotopes can be incorporated into peptides or proteins by growing cells in the presence of isotope-labelled metabolites and isolating the labelled peptides or proteins from these cells or cell culture supernatants. The isotopes can also be incorporated into peptides or proteins by other methods such as in vitro translation using isotope-labelled metabolites. Isotope-labelled metabolites can be amino acids, carbohydrates, fatty acids, inorganic salts such as phosphate, sulphate, etc. or other isotope-labelled substances. These isotope-labelled peptides or proteins are especially suitable for measuring methods using mass spectrometric methods.

Another kind of label especially suitable for mass spectrometric detection methods are isobaric labels. Different kinds of such labels have the same molecular weight, as long as these labels are intact and have not been fragmented in a mass spectrometer. This has the advantage, that mixtures of molecules labelled with different isobar labels can more conveniently be analyzed using mass spectrometric methods, as only a limited number of mass differences is present prior to analysis of the sample. Fragmentation of the isobar labels is done to determine the identity of the label, thereby identifying the identity of the molecule labelled with the isobar label. Preferably these labels are constructed in a way that upon fragmentation of the label in a mass spectrometer the fragments generated have masses, which masses usually are not obtained by fragmentation of peptides or proteins. Examples of such masses are masses between 114 and 120 Da. Generally isobaric labels should be selected, which generate masses, which are not generated in the same kind of samples, analyzed by the same mass spectrometric method in the absence of said isobaric labels.

In an alternative to directly using labelled amino acids in peptide synthesis, peptides and proteins generally can be labelled using amine-reactive reagents such as isothiocyanates, succinimidyl esters and carboxylic acids, sulfonyl chlorides, aldehydes, arylating reagents, etc. or thiol-reactive reagents such as iodoacetamides, maleimides, alkylhalides and arylating agents. Furthermore alcohols such as those present in serine, threonine or in carbohydrate moieties can be labelled by oxidizing with periodate to yield aldehydes that can be subsequently modified with a variety of amine or hydrazine derivatives. Alcohols from tyrosine sometimes can be reacted with sulfonyl chlorides or iodoacetamides. Alcohols in carbohydrate moieties furthermore can be labelled by reaction with dichlorotriazines. N-methylisatoic anhydride will convert ribonucleotides and other carbohydrate moieties to fluorescent esters with excitation/emission maxima of about 350/466 mm. Examples of fluorescent labels are fluorophores such as Alexa Fluor® 350, 405, 430, 488, 500, 514, 532, 546, 555, 568, 594, 610, 633, 635, 647, 660, 680, 700 and Alexa Fluor® 750, Aminomethylcoumarin (AMCA), Methoxy coumarin acetic acid, Bimane, BODIPY 493/503, 530/550, 558/568, 564/570, 576/589, 581/591, 630/650 and BODIPY 650/665, BODIPY FL, BODIPY TR; BODIPY TMR, Cascade Blue dye, Cascade Yellow dye, cyanine dyes such as Cy3, Cy5, Cy5.5, Cy7, Dansyl, Dapoxyl dye, Dialkylaminocoumarin, Eosin, Erythrosin, Fluorescein (FITC), Fluorescein-EX, 2′,4′,5′,7′-Tetrabromosulfonefluorescein, Naphthofluorescein, 2′,7′-Dichloro-fluorescein, 4′,5′-Dichloro-2′,7′-dimethoxy-fluorescein, 6-Carboxy-2′,4,4′,5′,7,7′-hexachlorofluorescein, succinimidyl ester (6-HEX, SE), 5-carboxyfluoresceine (5FAM), 5,6-carboxyfluoresceine (5(6)FAM), hydroxycoumarin, Malachite green, Marina Blue dye, methoxycoumarin, 7-nitro-4-benzofurazanyl (NBD), Oregon Green® 488, Oregon Green® 514, Pacific Blue, Pacific Blue dye, PyMPO, Pyrene, QSY 7, QSY 9, QSY 21, QSY 35, Rhodamine 6G, Rhodamine Green, Rhodamine Green dye, Rhodamine Red dye, Rhodamine Red, Rhodamine Red-X (RRX), X-rhodamine, Tetramethyl-rhodamine (TMR, TRITC), Lissamine rhodamine B, Texas Red, Texas Red dye, Texas Red-X. Most of these fluorophores are available from Molecular Probes, Eugene, Oreg., USA, and many are also available as kits intended for protein or peptide labelling. Furthermore fluorescent proteins or fragments or derivatives thereof such as green fluorescent protein, red fluorescent protein, blue fluorescent protein, renilla fluorescent protein, etc. can be used as labels. Examples of radioactive isotopes suitable as labels are phosphorous-32 or -33, sulphur-35, iodine-125, tritium, carbon-14, calcium-45, chromium-51 and all other known radioisotopes which can be coupled to nucleic acids and/or peptides or proteins. It is also possible to use matched pairs of fluorescent labels, which are suitable for fluorescence resonance energy transfer (FRET).

Examples of enzyme labels are horse-reddish-peroxidase, alkaline phosphatase, luciferase, galactosidase, etc. Suitable toxin labels are all kinds of toxins such as microbial toxins, toxic peptides or toxic small organic molecules, synthetic toxins or toxins used for medical purposes. An example of suitable dye labels is digoxigenin. Examples of particles as labels are particles of various particle diameters, preferably between 0.1 to 100 μm. Metal particles can be made from various substances, preferably heavy elements such as gold, silver, platinum or other suitable metals or alloys or mixtures thereof. Examples of magnetic particles are preferably made from all kinds of magnetic or magnetizable materials including iron, preferably iron oxides such as Fe₃O, Fe₂O₃, Fe₃O₄, etc. Examples of polymer particles are particles made from a polymer such as polyacryl amide, agarose, polypropylene, polystyrene, polytetrafluoroethylene, fluorescent polystyrene microspheres, etc. Various other labels, such as biotin, chitin, maltose or glutathione etc. as known in the art, can also be used.

Furthermore labels comprising certain additional nucleic acid or amino acid sequences subsequently termed label-sequences can be used. Label-sequences comprising nucleic acid sequences for example can be used for detection of the labelled substance for example by hybridization with a probe specific for the label-sequence or by detection of the label-sequence by polymerase chain reaction, preferably by quantitative polymerase chain reaction, preferably by real-time polymerase chain reaction, etc. Examples of label-sequences comprising amino acid sequences are his-tags, flag-tags, myc-tags or whole proteins or fragments thereof such as antibodies, glutathione S-transferase, streptavidin, chitin-binding protein, maltose binding protein, various lectins, etc. These labels can be detected by use of antibodies or by use of ligands binding to these labels such as protein A, protein G, protein Y, protein A/G, glutathione, biotin, chitin, maltose or other sugars etc. Label-sequences in general can also be used to capture the labelled peptide or protein by a binding agent specific for said label-sequence. Subsequently or during the capture process the labelled peptide or protein can be quantified. Labels can be at the N- or C-terminus or can be present internal in the amino acid sequence of a peptide and/or protein.

Reference Sample/Reference Value

According to the invention, the ability and/or specificity of a substance to modulate the activity of at least one protease is determined from a sample, i.e. test sample, comprising said substance, said at least one protease and a plurality of peptides and/or proteins. According to the invention, a “reference sample” may be used to correlate and compare the results obtained from a test sample. The composition of a “reference sample” is usually similar to a test sample but differs from the test sample in certain variables. In preferred embodiments of the methods of the invention a test sample and a reference sample differ in that the substance present in the test sample for determining its ability and/or specificity to modulate the activity of at least one protease is missing in the reference sample. In further embodiments of the methods of the invention, the reference sample contains a known modulator of the activity of said at least one protease, i.e. a substance the activity and/or specificity of which with respect to the modulation of proteolytic activity is known. This allows, for example, to draw conclusions whether the tested substance has a higher or lower modulatory activity and/or a same or similar or different specificity with respect to the modulation of proteolytic activity compared to the known modulator. Further knowledge about possible adverse side effects of said known substance or about patients subgroups best treated with said known substance can be used to draw further conclusions for the unknown substance, if the peptide pattern specific for the known and the unknown substance are compared.

In certain embodiments of the methods of the present invention, the reference sample and test sample might differ in their origin and/or composition, e.g. the test sample is derived from a diseased individual and the reference sample is derived from a healthy individual and it is tested whether the ability and/or specificity of a substance to modulate the activity of at least one protease is the same or different in both samples. These embodiments of the present invention allow for the screening of therapeutic substances having specific activity or higher activity in a diseased individual compared to a healthy individual.

The reference value can be determined empirically by measuring a sufficiently large number of samples. Preferably the reference value is determined by measuring at least 2, preferably at least 3, preferably at least 5, preferably at least 8, preferably at least 12, preferably at least 20, preferably at least 30, preferably at least 50, or preferably at least 100 samples.

Sample

The term “sample” according to the invention comprises any material which may be used for testing in the methods according to the invention. The sample may be derived from any source and may include components of different sources. As used herein, a “biological sample” is any sample obtained from or present in a biological system such as a living organism. Preferably, the sample is blood, serum, plasma, urine, liquor, bronchial aspirate, sputum, tissue extracts, cell extracts, cell culture medium. A biological sample may be derived from human beings and other primates, such as baboon, ape, monkey, etc.; economic animals, such as, bovine, caprine, swine, rabbit, murine, as well as pets, such as feline, canine, etc. More preferably, the sample is a blood, serum or plasma sample obtained from a patient. Preferably, natural sources for the samples and/or components thereof used in the methods of the present invention, in particular for the proteases and/or peptides and/or proteins used in the methods of the invention, are humans, animals, plants, microorganisms, in vitro cultured cells or tissues or tissue or cell culture medium. Preferably the individuals from which the samples or components thereof are obtained are small laboratory animals such as rats, mice, hamsters, guinea pigs, gerbils, rabbits, zebra fish, drosophila flies or larger mammals, preferably pigs, mini pigs, dogs, cats, apes or humans. It is possible to use tissues such as fat, muscle, blood, bone, intestine, pancreas, liver or tumour tissue or to use in vitro cultured primary cells or cell lines such as fat, muscle, blood, bone, intestine, pancreas, liver or tumour cells or cell culture medium from those primary cells or cell lines as samples or components thereof. The sample or components thereof can be a present in nature and can be obtained by methods known in the art such as isolation or purification techniques or can be obtained by use of various synthetic techniques such as chemical synthesis, enzymatical synthesis (for example in vitro translation), purification etc. Any kind of the above described materials, components thereof or combinations thereof can be used according to the invention.

Preferably, a sample according to the present invention or component thereof is or is derived from body fluid such as blood, plasma or urine, fat tissue, tumour tissue, etc. and is present in the natural environment of the living or dead organism.

Comparisons Between Samples

If the methods of the invention are to be applied to more than one sample, in particular to create a mean, the samples can differ from one another, in particular regarding the time point they have been taken from the source which may be the same or different or the time the substance being tested is allowed to contact at least one protease. The difference in time can be more than 1 second, more than 20 seconds, more than 1 minute, more than 5 minutes, more than 10 minutes, more than 30 minutes, more than 1 hour, more than 2 hours, more than 6 hours, more than 12 hours, more than 1 day, more than 3 days, more than a week, more than a month, or more than a year. Such samples are suitable to perform the methods of the invention in a time-dependent manner.

If the methods of the invention are to be applied to more than one sample, the samples or components thereof can also differ regarding the source from which they have been obtained. For example the samples or components thereof may be derived from different tissue culture plates, different individuals, different kinds of tissues or cells, different species or organisms of different genetic backgrounds (e.g. genetic knock-out versus wild-type, or different ethnic backgrounds), etc. The samples or components thereof may differ regarding the sex, age, body mass of the organism, from which they originate, i.e. are derived, or regarding the exposure of the organism, from which the samples or components thereof originate, to chemicals, alcohol, cigarette smoke, etc. The samples or components thereof may differ regarding the physical activity, exposure to environmental factors such as access to food, sugar-rich diets, fat-rich diets, protein-rich diets, water, etc. or air pollution, light, humidity, temperature, noise, radiation, psychological stress, etc. of the organism from which the sample or components thereof originate. The samples or components thereof may originate from individuals having or not having a disease or other condition, from organisms having different diseases or other conditions or from organisms having different stages of diseases or other conditions. Different stages of a disease or other condition include the time points before, during or after the occurrence or first diagnosis of said disease or other condition. Other conditions among others include pregnancy and fertility. The samples or components thereof may differ regarding their occurrence within a living organism such as serum, plasma, whole blood, urine, tissue extracts, etc., origin, e.g. from individuals treated or not treated with particular compounds or compositions, in particular compounds or compositions to be tested for their ability and/or specificity to modulate the activity of at least one protease in the methods of the invention. If individuals, cell cultures or samples are treated with compounds or compositions they may be treated for different time intervals, or with different concentrations, or with different compounds or compositions, or with mixtures of two or more compounds or compositions at the same time or at different times, or under different experimental conditions such as temperature, light, humidity, access to nutrients, etc. Samples or components thereof may also differ by various combinations of the above described features.

Preferably, the results obtained using different samples or components thereof are compared to each other.

Peptide and/or Protein Pattern

The methods of the invention are performed by generating protein and/or peptide patterns which patterns comprise at least two distinct signals and preferably comparing protein and/or peptide patterns generated. The protein and/or peptide patterns preferably include signals of substrates of at least one protease and/or products formed by the activity of at least one protease. A comparison of protein and/or peptide patterns may reflect different amounts of substrates of at least one protease and/or products formed by the activity of at least one protease and thus, be indicative for different activities of at least one protease in the samples on the basis of which the patterns were established and/or source from which the samples are derived, which in turn could be indicative for the ability and/or specificity of a compound or composition to modulate the activity said at least one protease.

Such protein and/or peptide pattern preferably comprises two or more, preferably 4 or more, more preferably 6 or more, 8 or more, 10 or more, 15 or more, even more preferably 20 or more, 30 or more, 40 or more, 50 or more, 60 or more, 70 or more, 80 or more, 90 or more, 100 or more, 120 or more, 140 or more, 160 or more, 180 or more, 200 or more, 250 or more, 300 or more, 400 or more, 500 or more signals for peptides and/or proteins. The upper limit of said signals is not particularly restricted as long as the number of said signals and thus, the number of peptides and/or proteins determined does not exceed the resolution limit of the assay system and the available computing power to calculate the correlations. An upper limit may be for example not more than 20, 40, 60, 80, 100, 150, 200, 250, 300, 400, 450, 500, 1000, 2000, 5000 or 10000 signals depending on the assay system used in the methods of the invention. In certain embodiments of the methods of the invention, only a part or fraction of the peptides and/or proteins resulting from proteolytic cleavage is determined which preferably results in less signals in a protein and/or peptide pattern. In these embodiments the peptides and/or proteins to be determined may be subjected to separation means such as chromatographic separations before being determined. For evaluation of such patterns preferably at least one of the following mathematical methods to calculate correlations is used: “Pearson Product-Moment Correlation”, “Spearman's Rank-Order Correlation”, “Kendall's Tau”, “Kendall's Coefficient of Concordance”, “Goodman and Kruskal's Gamma”, “Manhattan distance”, “Euclidean distance” and “Minimal Spanning Tree Diameter”. Preferably “Spearman's Rank-Order Correlation” is used.

Correlations can be calculated for two or more peptides or proteins, between one or more peptides or proteins and additional meta data available, such as protease activity, concentrations of compounds or compositions, preferably protease inhibitors or activators. Further suitable meta data among others are absence or presence of a disease or other conditions, state of the disease, other clinical parameters such as blood presure, body temperature, medication for disease treatment, age, sex, exporsure to environmentel conditions such es temprature, stress, etc. The modulation of the activity of at least one protease according to the invention can be reversible or irreversible or mixed, specific or non-specific, specific for particular protease families within a sample or specific for individual proteases or subgroups of proteases within a sample.

The protein and/or peptide patterns generated by the methods of the invention may comprise signals for peptides and/or proteins wherein one or more signals are stronger and other signals are weaker compared to a reference sample. In situations where the reference sample does not contain the compound or composition to be tested which is present in the test sample such pattern may be indicative for the fact that the compound or composition modulates the specificity of one or more proteases present in the sample, increases the activity of one or more proteases present in the sample (in this situation some of the peptides and/or proteins may represent substrates for and others may represent products formed by the activity of at least one protease) or decreases the activity of one or more proteases present in the sample (in this situation some of the peptides and/or proteins may represent substrates for and others may represent products formed by the activity of at least one protease) or decreases the activity of one or more proteases present in the sample while it increases the activity of other proteases present in the sample or a combination thereof.

“Different peptides and/or proteins” according to the invention relate to peptides and proteins which are not identical in their chemical composition, in particular sequence, structure, activity, specificity etc. such as different fragments of the same or of different precursor proteins or peptides, the same peptide or protein with different posttranslational, or chemical, or other modifications, etc.

Modulators of Proteases

The methods of the present invention allow for the identification of modulators, in particular inhibitors or activators, of all kinds of proteases, for example proteases belonging to aspartic peptidases, cysteine peptidases, glutamic peptidases, metallo peptidases, serine peptidases, threonine peptidases, amino peptidases, carboxypeptidases, endopeptidases, exopeptidases, specifically angiotensin converting enzyme (ACE), ACE2, renin, neutral endopeptidase (NEP) (hypertension), HIV-1 protease, HIV-2 protease, factor Xa, factor VIIa, factor XIIIa (thrombosis), interleukin-1 converting enzyme (ICE) (inflammatory diseases), dipeptidyl peptidase 4 (DPP4) (type II diabetes), alpha-, beta-, gamma-secretase, caspase (Alzheimer's disease), cathepsin K (osteoporosis), Matrix metalloproteinases (MMPs), proteosome protease (cancer), NS3 protease (HCV).

If a compound or composition has been determined to have the ability to modulate the activity of at least one protease, preferably by the methods of the invention, it is possible to further characterize and specify said ability by changing certain test parameters or variables, e.g. using different types of samples, using different peptides and/or proteins, using different proteases, inhibiting the activity of certain proteases within the sample, e.g. by adding known inhibitors etc. Thus, the methods of the invention can be performed more than once in a simultaneous and/or sequential manner such as in a repetitive manner, whereby certain test parameters or variables are varied to characterize a compound or composition in more detail, in particular obtain detailed knowledge about the specificity of its modulation of proteolytic activity.

Compound or Composition

The term “compound or composition” according to the invention relates to all kinds of substances including small organic or inorganic molecules, chemically, enzymatically or recombinantly synthesized molecules, such as nucleic acids, peptides, proteins, carbohydrates or lipids, synthetic molecules or molecules found in nature or molecules isolated from materials or organisms found in nature, as well as molecules found in nature with altered structures or with sequences comprising deletions, insertions and mutations of the sequence and combinations or mixtures thereof. Compositions according to the invention comprise two or more compounds or at least one compound and additional substances such as saline, diluents, solvents, pH buffering agents, osmolarity or viscosity regulating agents, additives for preservation, staining, flavouring or scenting, filling substances, anti-microbial, anti-fungal or other preserving agents, substances protecting the compound or composition form light, heat, freezing, or other environmental factors, substances preventing aggregation or sticking of compounds to the container, formulations necessary for specific routes of applications such as gastric acid resistant capsules, or plasters for transdermale application of compounds or compositions etc.

Therapeutic Application

The methods of the invention allow for the identification of compounds or compositions which are therapeutically applicable. The expression “therapeutically applicable” means that the compounds or compositions are effective against a disease or condition, i.e. may be used to reduce the risk of an individual treated with said compound or composition to acquire said disease or condition, or heal said disease or condition or stop or slow down the progression of said disease or condition in an individual afflicted therewith.

Peptides and Proteins

The term “peptide” refers to substances consisting of two or more, preferably 3 or more, preferably 4 or more, preferably 6 or more, preferably 8 or more, preferably 10 or more, preferably 13 or more, preferably 16 more, preferably 21 or more amino acids joined covalently by peptide bonds. The term “protein” refers to large peptides, preferably to peptides with at least 160 amino acids, but in general the terms “peptides” and “proteins” are synonyms and are used in this application as synonyms. The terms “peptide” and “protein” according to the invention also include substances containing not only amino acids, but also non-amino acid constituents and include substances containing only peptide bonds as well as substances also containing other bonds, e.g. ester, thioether or disulfide bonds.

Peptides and variants thereof having less than about 100 amino acids, and generally less than about 50 amino acids, may be generated by synthetic means, using techniques well known to those of ordinary skill in the art. For example, such peptides may be synthesized using any of the commercially available solid-phase techniques, such as the Merrifield solid-phase synthesis method, where amino acids are sequentially added to a growing amino acid chain. See Merrifield, 1963, J. Am. Chem. Soc. 85, 2149-2146. Equipment for automated synthesis of polypeptides is commercially available from suppliers such as Perkin Elmer/Applied BioSystems Division (Foster City, Calif.), and may be operated according to the manufacturer's instructions.

The peptides and variants described herein can be obtained, for instance, by designing peptides or proteins which include a protease recognition sequence and thus, may be substrates for at least one protease.

Within certain specific embodiments, a peptide or protein may be a fusion peptide or fusion protein that may comprise multiple protease recognition sequences. A fusion partner may, for example, assist in expressing the peptide or protein (an expression enhancer) at higher yields than the native recombinant peptide. Other fusion partners may be selected so as to increase the solubility of the peptide or protein or to enable the peptide or protein to be targeted to desired intracellular compartments. Still further fusion partners include affinity tags, which facilitate purification and/or detection of the peptide or protein.

Fusion peptides or fusion proteins may generally be prepared using standard techniques, including chemical conjugation. Preferably, a fusion peptide or fusion protein is expressed as a recombinant protein, allowing the production of increased levels, relative to a non-fused peptide or protein, in an expression system. Briefly, DNA sequences encoding the peptide or protein components may be assembled separately, and ligated into an appropriate expression vector. The 3′ end of the DNA sequence encoding one peptide or protein component is ligated, with or without a DNA sequence encoding a peptide linker, to the 5′ end of a DNA sequence encoding the second peptide or protein component so that the reading frames of the sequences are in phase. This permits translation into a single fusion peptide or fusion protein that retains the biological activity of both component peptides or proteins.

A peptide linker sequence may be employed to separate the first and second peptide or protein components by a distance sufficient to ensure that each peptide or protein folds into its secondary and tertiary structures. Such a peptide linker sequence is incorporated into the fusion peptide or fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second peptides or proteins; and (3) the lack of hydrophobic or charged residues that might react with the peptide or protein functional epitopes. The linker sequence may generally be from 1 to about 50 amino acids in length. Linker sequences are not required when the first and second peptides or proteins have non-essential N-terminal amino acid regions that can be used to separate the functional domains and prevent steric interference.

Modifications of Peptides and Proteins

Modifications of peptides or proteins described herein may comprise modifications due to posttranslational modifications, chemical modifications, enzymatical modifications and modifications due to other mechanisms. Examples of possible modifications include but are not limited to: glycosylation, phosphorylation, sulphatation, pyroglutamate modification, cystein-disulfide bridges, methylation, acetylation, acylation, farnesylation, formylation, geranylgeranylation, biotinylation, stearoylation, palmitylation, lipolyation, C-mannosylation, myristoylation, amidation, deamidation, methylation, demethylation, carboxylation, hydroxylation, iodination, oxidation, pegylation, prenylation, ADP-ribosylation, addition of lipids, of phosphatidylinositol, of glycosylphosphatidylinositol (GPI)-anchor, of pyridoxal phosphate, modification of cysteine residues resulting in carboxyamidomethylcysteine, resulting in carboxymethylcysteine, or resulting in pyridylethylcysteine, modification of lysine residues resulting in liponic acid, modification of glutamic acid resulting in pyroglutamic acid, etc.

Modifications of peptides or proteins described herein may comprise unusual amino acids, chemically or enzymatically modified amino acids etc. including, but not limited to: alpha amino butyric acid, beta amino butyric acid, beta amino iso-butyric acid, beta alanine, gamma butyric acid, alpha amino adipic acid, 4-amino benzoic acid, amino ethyl cysteine, alpha amino penicillanic acid, allysine, 4-carboxy glutamic acid, cystathionine, carboxy glutamic acid, carboxy amido methyl cysteine, carboxy methyl cysteine, cysteine acid, citrulline, dehydroalanine, di-amino butyric acid, dehydro amino-2-butyric acid, ethionine, glycine-proline di-peptide, 4-hydroxyproline, hydroxylysine, hydroxyproline, homoserine, homo cysteine, histamine, iso-valine, lysinoalanine, lanthionine, norvaline, norleucine, ornithine, 2-pipiridine-carboxylic acid, pyroglutamic acid, pyrrolysine, proline-hydroxy proline di-peptide, sarcosine, 4-selenocysteine, syndesine, thioproline, etc. Further examples can be found in databases such as the “Delta Mass” database searchable at the website of the ABRF, the “Association of Biomolecular Resource Facilities”: http://www.abrf.org/index.cfm/dm.home?AvgMass=all.

Peptidomimetics of Peptides and Proteins

Peptides and proteins described herein can be prepared or synthesized to represent partially or completely peptidomimetics. This allows to design peptides with more defined properties. For example D-amino acids could be used to prevent proteolysis. “Peptidomimetics” according to the invention are structures, which mimic the function of amino acids or amino acid sequences. “Peptidomimetics” or “mimetics of peptides” often have additional properties such as increased resistance to proteolysis. Peptidomimetics can replace some or all peptide bonds by other kinds of covalent bonds which can connect amino acids. Peptidomimetics among others can comprise modifications of amino acids such as alpha-C alkylation, alpha-N alkylation, etc, dipeptide analogues (two amino acid side chains which are connected by covalent bonds) or modifications of the peptide backbone, especially change from L- to D-amino acids, inverse N- to C-sequence, amide bond isosteres, etc. Further examples of peptidomimetics are spiegelmers® (NOXXON Parma AG, Berlin, Germany) or beta amino acids.

Nucleic Acids, Complementary and Degenerated Nucleic Acids and Derivatives

With nucleic acids or nucleic acid sequences according to the invention are meant all kinds of deoxyribonucleic acids (DNA) and ribonucleic acids (RNA) or combinations thereof regardless if these nucleic acid molecules are single or double-stranded, linear, circular or branched. Examples of nucleic acid molecules are genomic DNA, cDNA (complementary DNA), mRNA (messenger RNA), recombinantly produced nucleic acid molecules, chemically synthesized nucleic acid molecules or enzymatically generated nucleic acid molecules for example by use of polymerase chain reaction (PCR) or nucleic acid molecules purified from natural sources such as prokaryotic or eukaryotic cells, viruses, tissues, organs or whole organisms such as bacteria, yeast, insects, worms, etc. using methods known in the art. Furthermore nucleic acid molecules according to the invention can be partially or completely represent derivatives of nucleic acids which derivatives comprise nucleotides or nucleotide-like molecules not found in nature such as phosphorothioates, peptide nucleic acids (PNAs), N3′,P5′-phosphoramidates, morpholino phosphoroamidates, 2′-O-methoxyethyl nucleic acids, 2′-fluoro-nucleic acids, arabino-nucleic acids, locked nucleic acids (LNA, ribonucleotides containing a methylene bridge that connects the 2′-oxygen of ribose with the 4′-carbon), etc.

Methods to Determine Peptides and Proteins

Generally all methods suitable to detect and analyse peptides and proteins can be used in the methods of the invention and can be used to determine the presence, absence or quantity of the peptides and proteins described herein, and thus can be used to determine the activity and/or specificity of a protease. Preferably mass spectrometric methods, protein chip assays, immunological and molecular biology methods can be used for this purpose.

Preferably, in the methods of the invention peptides and/or proteins of a molecular weight not more than 100 kDa, preferably not more than 80 kDa, or 70 kDa, 60 kDa, 50 kDa, 40 kDa, 30 kDa, 20 kDa, 10 kDa and preferably at least 5 kDa or 10 kDa are determined.

In certain embodiments of the methods of the invention, high-molecular weight peptides or proteins and other biopolymers which might interfere with the measurement are removed from the sample before measurement. Suitable methods for this among others are native, denaturing or 2-D gel electrophoresis, liquid chromatography such as anion or cation exchange chromatography, metal chelate affinity chromatography, affinity chromatography, reversed phase chromatography, gas chromatography, capillary chromatography, thin layer chromatography, mass spectrometry, precipitation techniques such as trichoric acetic acid or trifluouric acid or ethanol precipition, immune precipitation, liquid phase extraction techniques, filtration and other molecular size discriminating techniques such as ultrafiltration using membranes with for example size exclusion limits of 3, 5, 10, 30 or 50 kilo Dalton.

The sample tested may be divided into several fractions for determination of the peptides and/or proteins and/or the samples analyzed with different measuring arrangements.

Suitable mass spectrometric methods among others are matrix assisted laser desorption ionisation (MALDI), continuous or pulsed electrospray ionization (ESI), SELDI mass spectrometry, FAB mass spectrometry and MS/MS mass spectrometry and related methods such as ionspray or thermospray or massive cluster impact (MCI). The ion sources can be matched with detection formats including linear or non-linear reflection time-of-flight (TOF), single or multiple quadrupole, single or multiple magnetic sector, fourier transform ion cyclotron resonance (FTICR), ion trap, and combinations thereof, e.g. ion-trap/time-of-flight. For ionisation, numerous matrix/wavelength combinations (MALDI) or solvent combinations (ESI) can be used. Other mass spectrometric methods suitable are for example fast atom bombardment (FAB) mass spectrometry, Surface Enhanced Laser Desorption/Ionisation (SELDI) mass spectrometry, isotope coded affinity tag (ICAT) mass spectrometry, or affinity mass spectrometric methods.

Furthermore suitable immunologic methods among others are enzyme linked immuno assays (ELISA), sandwich, direct, indirect, or competitive ELISA assays, enzyme-linked immunospot assays (ELISPOT), radio immuno assays (RIA), flow cytometry assays (FACS=fluorescence activated cell sorting), immunohistochemistry, Western blot, fluorescence resonance energy transfer (FRET) assays, protein-chip assays using for example antibodies, antibody fragments, receptors, ligands, or other binding agents specific for the peptides or proteins described herein.

Further methods such as nuclear magnetic resonance (NMR), fluorometry, colorimetry, radiometry, luminometry, or other spectrometric methods, liquid chromatography, capillary chromatography, thin-layer chromatography, plasmon resonance (BIACORE), one- or two-dimensional gel electrophoresis, etc. can be used to detect the peptides and proteins described herein.

### EXEMPLIFICATION OF THE INVENTION

The following examples are intended to illustrate the invention, however they are not meant to limit the scope of the invention in any way.

### Example 1

### In Vivo DPP4 Inhibition in Rats

The DPP4 inhibitor AB192 (bis(4-acetamidophenyl)-1-(S)-prolylpyrrolidine-2-(R,S)-phosphonate) (J. Med. Chem., 1999, 42:1041-52) was kindly provided by Ingrid De Meester (University of Antwerp, Campus Drie Eiken, Department of Pharmaceutical Sciences, Universiteitsplein 1, B-2610 Wilrijk-Antwerpen). AB192, an irreversible inhibitor of DPP4, was used at 0.32, 1.00 and 3.2 mg/kg body weight. The control inhibitor (Contr.In.) was used at 0.3, 1.0 and 3.0 mg/kg body weight. Eight week old Wistar rats (315-320 g body weight) received either of the inhibitors (8 animals for each inhibitor concentration of each inhibitor) or were treated with vehicle (50 mM sodium phosphate buffer; pH 7.4) (4 animals). The rats received the inhibitors or vehicle by subcutaneous injection into the fat tissue of the neck using a 27-G needle. Blood samples (100 μl) for DPP4 activity measurements were drawn from the tail vein before injection and at the end of the experiment 1 h after injection of the DPP4 inhibitors. At this point, the rats were anaesthetised using a mixture of ketamin (9 mg/100 g body weight) and rompun (0.2 mg/100 g body weight). Additionally, rats received a short burst of ether for quick anaesthesia. The thorax was opened immediately and 4 to 6 ml of blood taken from the right ventricle into ethylenediaminetetraacetic acid (EDTA) laminated syringes and transferred into a 15 ml tube filled with EDTA at a final concentration of 2.5 mg/ml.

### Example 2

### Measurement of DPP4 Activity

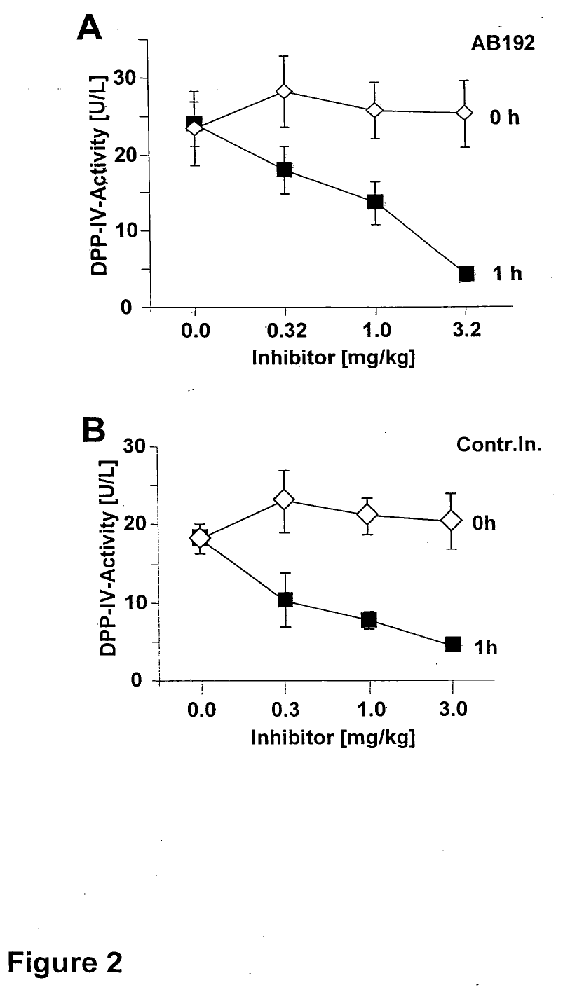

Blood samples taken from the tail vein were immediately centrifuged (2000×g; 10 min at room temperature) and plasma stored at −20° C. At the day of the measurement 10 μl of each sample was pipetted in duplicates directly in a 96-well microtiter plate placed on ice. After all samples and standards (0-0.5 mU/ml purified DPP4 enzyme from porcine kidney; Sigma-Aldrich Chemie GmbH, Munich, Germany) were added, the plate was placed on a 37° C. heating block and 40 μl freshly prepared substrate solution (500 μM G-P-4 nitroanilide diluted in 0.4 mM HEPES buffer) added to each well. The 96-well plate was read immediately using a Tecan Genios microplate reader (Tecan Deutschland GmbH, Crailsheim, Germany) and measurements were taken at 37° C. over a period of time of 1 h at a wavelength of 405 nm. From the measured values the blank signal (plasma+HEPES buffer) was subtracted and data calculated as Units/litre (U/l). The results of the measurements of the DPP4 activity measured in plasma of rats treated with two different inhibitors (AB192 and Contr.In.) at various concentrations and in plasma of control rats (treated with vehicle) is shown in FIG. 2. Both inhibitors tested inhibited the activity of DPP4 in vivo in rats in a dose dependent manner.

### Example 3

### Rat Plasma Preparation for Peptide Display

Rat blood samples were consecutively centrifuged at room temperature for 10 min at 2000×g and for 15 min at 2500×g. The resulting platelet poor plasma was stored at −80° C. until analysis. For reduction of the protein load of the plasma, the plasma sample was diluted 1:4 with 8 M guanidinium chloride solution at room temperature and for quality control purposes a peptide standard mix was added resulting in a final concentration of 300 to 800 pM of each individual standard peptide added. The peptides added were: substance P, somatostatin-14, neurotensin, renin substrate (porcine), adrenocorticotropic hormone (ACTH)(1-17), big endothelin-1(19-38), ACTH(18-39), beta-Endorphin(6-31), adrenocorticotropic hormone(1-24), calcitonin (human), insulin B chain oxide (bovine), ACTH(7-38), pro34-neuropeptide Y (porcine) and growth hormone-releasing factor (GRF)(1-44). All Peptides or proteins represent the human sequence, if not indicated to the contrary and the numbers in brackets following some of the peptides indicate the amino acid positions of the complete sequence including said peptide. Prior to use, the ultrafiltration devices are rinsed with distilled water. Ultrafiltration was done using Amicon Ultra filter devices with a molecular weight cut-off of 50 kDa (Millipore, Bedford, USA), which were centrifuged according to the manufacturer's instructions at 4000×g for 60 min at room temperature in a swinging bucket rotor. Finally, the pH of the filtrate was adjusted to pH 2 to 3 using concentrated HCl (30% v/v). If the sample (filtrate) was not directly further processed, it was stored at −80° C. A 750 μl-equivalent of plasma was used per chromatographic run.

### Example 4

### Liquid Chromatography of the Samples

The separation of peptides and/or proteins was done using a Source 5RPC, 4,6×150 mm reverse phase chromatography column (Amersham Biosciences Europe GmbH, Freiburg, Germany). Buffer A was 0.06% (v/v) (volume per volume) trifluoroacetic acid (TFA) in distilled water and buffer B was 0.05% (v/v) TFA, 80% (v/v) acetonitrile in distilled water. The chromatography took place at 33° C. using a HP 1100 HPLC with a micro flow cell both supplied by Agilent Technologies, Böblingen, Germany. The samples were diluted to 5.5 ml using a 0.1% (v/v) stock solution of trifluoroacetic acid. Prior to chromatography, the samples were centrifuged at 4° C. and 15 000×g for 10 minutes and finally 5 ml of sample were loaded onto the chromatography column representing an equivalent of 750 μl plasma. Buffers A and B were mixed in various ratios during chromatography and in the following only the amount in percentage of buffer B is stated. The amount in percentage of buffer A is 100% minus % buffer B (v/v). The chromatography conditions were as follows:
5% buffer B for 1 min,
a linear gradient from 5 to 50% buffer B during the next 44 min,
a linear gradient from 50 to 100% buffer B during the next 4 min,
100% buffer B for the next 4 min.
   The flow rate was 500 μl/min and 96 fractions each containing 0.25 ml were collected during the gradients from 5 to 100% buffer B.

### Example 5

### Mass Spectrometry of the Samples

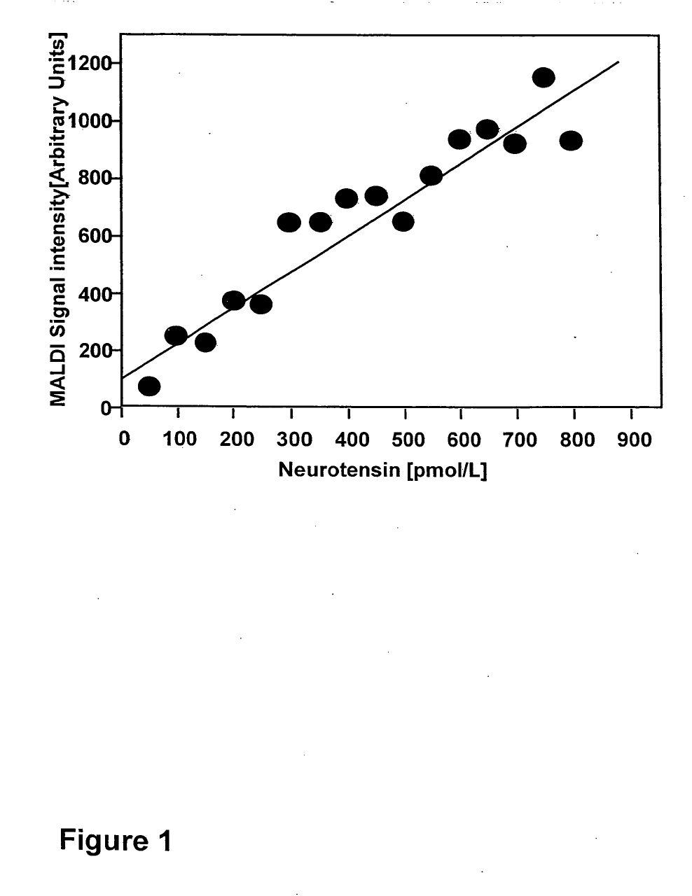

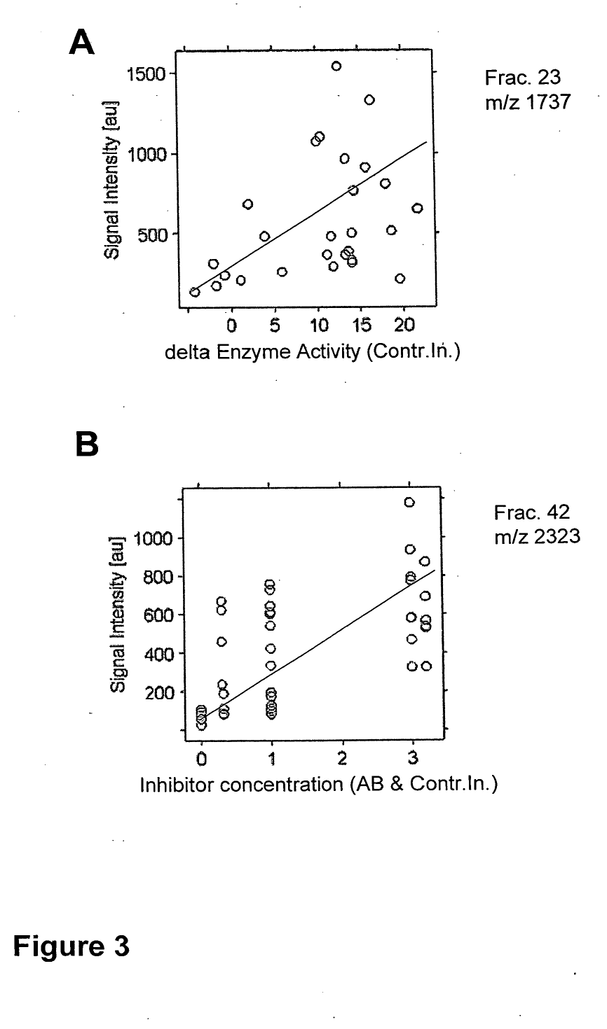

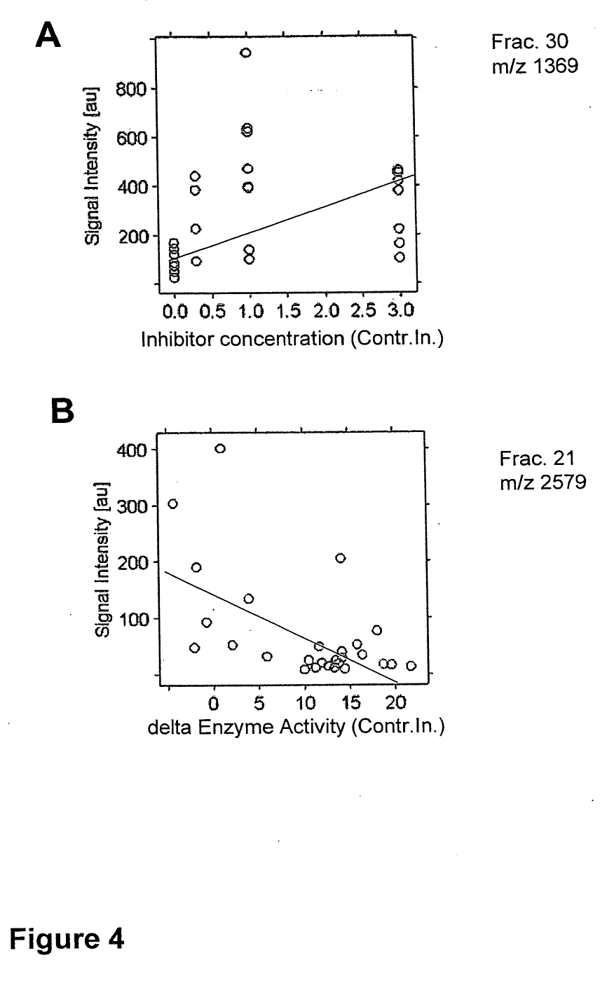

For mass spectrometric analysis, typical positive ion spectra of peptides were measured using a MALDI-TOF (matrix-assisted laser desorption ionization time of flight) mass spectrometer. For MALDI sample preparation alpha-cyano-4-hydroxycinnamic acid was used as matrix and 6-desoxy-1-galactose was used as co-matrix, dissolved in acetonitrile containing 0.1% TFA. A lyophilized equivalent obtained by reverse phase chromatography corresponding to 7.5 μl plasma spotted onto the mass spectrometric carrier plate was used to measure the peptides and/or proteins. The fractionated, lyophilized sample was dissolved in 15 μl of a matrix solution. This matrix solution contained 10 g/L α-cyano-4-hydroxycinnamic acid and 10 g/L L(−)fucose dissolved in a solvent mixture consisting of acetonitrile, water, trifluoroacetic acid (TFA) and acetone in a ratio of 49:49:1:1 by volume (v/v). 0.3 μl of this sample solution was transferred to a MALDI carrier plate, and the dried sample was analyzed in a Voyager-DE STR MALDI mass spectrometer from PerSeptive Biosystems. The measurement took place in linear mode with delayed extraction. A MALDI-TOF mass spectrometer can be employed to quantitatively determine peptides and/or proteins such as, for example, the peptides and proteins described herein if these peptides and/or proteins are present in a concentration which is within the dynamic measurement range of the mass spectrometer, thus avoiding detector saturation. The ability of MALDI mass spectrometry to quantitatively determine individual peptide and/or protein mass signals was confirmed by adding known concentrations (e.g. 50 to 800 pmol) of peptides, such as neurotensin, into complex biological samples such as plasma samples. Subsequently, these samples were separated by reversed phase chromatography and those fractions containing the added peptides were analyzed by mass spectrometry as described in the examples. There is a linear correlation between the MALDI mass spectrometry mass signal intensity of, for example, the neurotensin peptide and the amount of the neurotensin peptide added to the plasma sample, indicating that MALDI mass spectrometry is suitable to quantitatively determine peptides and/or proteins in complex samples such as plasma (FIG. 1). The correlation of the mass signal of exemplary peptides listed in Table 1 and of the DPP4 activity measured in the same samples or of the amount of inhibitor applied to the rats used in the experiment is shown in FIGS. 3 and 4 and in Table 1.

### Example 6

### Peptide Identification

Detection of concentration differences of individual peptides and/or proteins was achieved by calculation of subtractive peptide display maps and correlation analysis. Selected peptides, which appeared in peptide display maps were analyzed by ESI-qTOF sequencing (PE Applied Biosystems, Framingham, USA). Peptide ions were selected in the mass spectrometer on the basis of their specific m/z (mass/charge) values in a manner known to the skilled worker. These selected ions were then fragmented by supplying collision energy (20-40 eV) with an collision gas, e.g. helium or nitrogen, and the resulting fragments of the peptides were detected in the mass spectrometer in an integrated analysis unit, and corresponding m/z values were determined (principle of tandem mass spectrometry). The fragmentation behaviour of peptides and/or proteins enables the unambiguous identification of the peptides and/or proteins. The mass-spectrometric analysis can be done for example by Quadrupol-TOF (time of flight) sequencing (QStar-Pulsar model) or ESI-qTOF sequencing (both from PE Applied Biosystems, Framingham, USA). The resulting peptide fragment spectra were detected using nanoSpray in the product ion scan mode (spray voltage 950 V, collision energy 20-40 eV). Up to 200 scans per sample were accumulated. Charge state deconvolution was performed using the Bayesian reconstruct tool of the BioAnalyst program package (PE Applied Biosystems), and deisotoping was achieved by means of the Voyager 5.1 software (PE Applied Biosystems). The mass spectra were saved in a MASCOT generic file format, and submitted to the MASCOT19 database search engine (Matrix Science, London, UK). Searched databases were SwissProt (Version 39.6, www.expasy.ch) and MSDB (Version 010721, EBI, Europe). In addition, this peptide/protein sequencing method allows the identification of amino acid modifications such as phosphorylation, acetylation or hydroxylation.

### Example 7

### Data Analysis

Subsequently to fractionation as described in Example 4, each fraction was individually analyzed by MALDI mass spectrometry as described in Example 5 resulting in 96 mass spectra for each sample. These 96 mass spectra were electronically combined resulting in a so called differential peptide display. The x-axis of these differential peptide displays depicts the molecular mass, the y-axis depicts the fraction number and the colour intensity represents the mass spectrometric signal intensity. The data of differential peptide displays may be pre-processed by minimizing background noise, and outliers may be detected, e.g. visually or by means of principal component analysis (Pirouette 3.0, Infometrix Inc, Wash., USA), and excluded from further analysis.

Peptide signals, whose concentration and thus, mass spectrometric signal intensity correlated with an endpoint such as the concentration of the applied protease inhibitor, were in focus of investigation: Various correlations were performed correlating the mass spectrometric signal intensities with meta data such as the concentration of the protease inhibitor used or with the change in protease activity (“delta DPP4 activity”=“before/after change in DPP4 activity”) determined in rat plasma after administration of the protease inhibitor.

Mass spectrometric signals correlating with
A) the concentration of AB192 and with the concentration of the control inhibitor (Contr.In.) (n=49, |r|>0.5)
B) the concentration of AB192, but not with the concentration of the control inhibitor (n=29, |r|>0.59)
C) the concentration of the control inhibitor, but not with the concentration of AB192 (n=27, |r|>0.61)
D) the “before/after change in DPP4 activity” in the presence of AB192 and with the “before/after change in DPP4 activity” in the presence of the control inhibitor (n=49, |r|>0.5)
E) the “before/after change in DPP4 activity” in the presence of AB192, but not with the “before/after change in DPP4 activity” in the presence of the control inhibitor (n=29, |r|>0.59)
F) the “before/after change in DPP4 activity” in the presence of the control inhibitor, but not with “before/after change in DPP4 activity” in the presence of AB192 (n=31, |r|>0.61)
   were searched. This correlation analysis can be performed, for example, by means of Spearman correlation, e.g. with R-package, version 2.0 (R Foundation for Statistical Computing, Vienna, Austria) or S-plus, version 6.0 (Insightful AG, Reinach, Switzerland), etc.

In order to exclusively obtain signals within the 95% confidence interval, which depends on the number of samples analyzed, different threshold values of Spearman's correlation coefficients (|r|) were used (see points A to F above) to determine the significant peptide or protein coordinates. Furthermore, the threshold values of change of signal intensities of the control group (no inhibitor added) to the groups having received inhibitors, should be determined empirically, and was in this example 18 arbitrary units.

Subsequently, redundant signal coordinates were removed resulting in the remaining list of signal coordinates shown in Table 1. Redundant signals are, for example, signal coordinates with the same m/z values in directly adjacent fractions, which most likely represent the same peptide or protein which by chance eluted in two or more adjacent fractions, or m/z values representing multiples of a m/z value (e.g. 2050 and 4100) and which are present in the same or directly adjacent fractions (most likely representing multiple charged versions of an ion of the same molecule). Each signal coordinate is only listed once in Table 1. If the same signal coordinate (fraction and m/z value) was found in more than one of the groups A to F, this fact is indicated in Table 1.

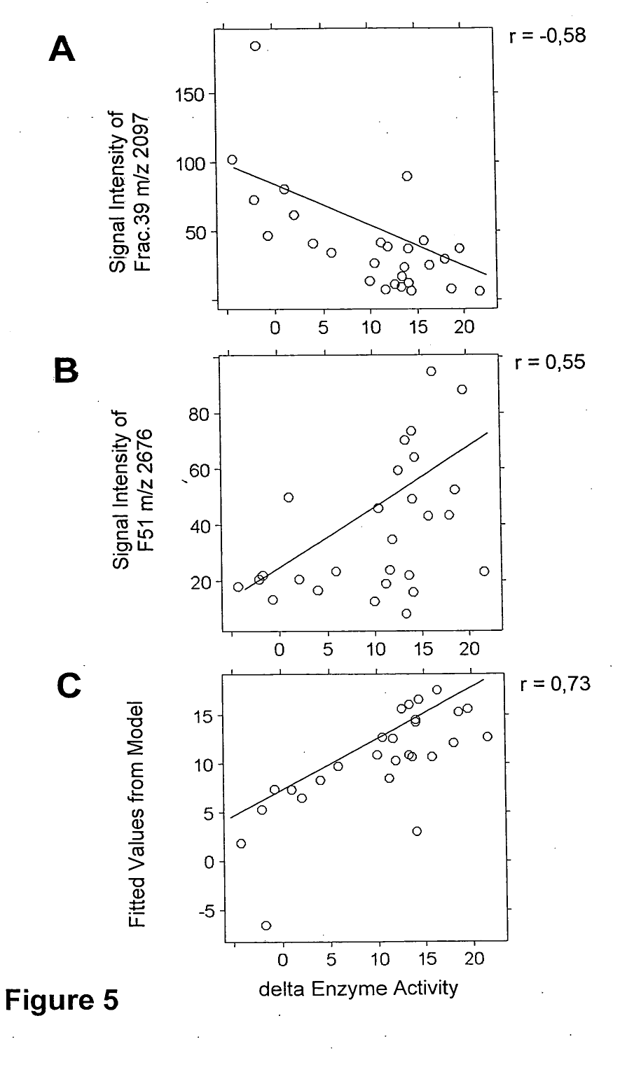

In addition, networks of peptides and/or proteins were calculated. This allows, for example, to identify panels of peptides and/or proteins, which enable to better reflect the biological impact of the inhibitors (FIG. 5/Table 2). Combining two peptides and/or proteins in these panels increased the reliability of the prediction (e.g. concentration of inhibitor, “before/after change in DPP4 activity”) possible by evaluating these peptide/protein panels. These panels were determined by comprehensively combining peptide and/or protein signals to pairs of peptide and/or protein signals. In a “linear model”, the signal intensities of the peptide and/or protein pairs were treated as independent variables, and the endpoint DPP4 concentration (or change in DPP4 activity) was treated as the dependent variable. The “linear model” was then calculated (e.g. with R-package, version 2.00 (R Foundation for Statistical Computing, Vienna, Austria) or S-plus, version 6.0 (Insightful AG, Reinach, Switzerland), etc.). Those models with pairs of peptide and/or protein signals, which resulted in a better prediction of the endpoint compared to separate single peptide and/or protein signals, as indicated by an increased correlation coefficient in row “Panel 1+2” of Table 2, are putative panels of peptides and/or proteins.

In addition to panels of peptides and/or proteins, surrogate networks of peptides and/or proteins can also be predicted, which peptides and/or proteins can replace each other, i.e. the peptides and/or proteins are surrogates of each other. Using this method it is, for example, possible to identify peptides and/or proteins which have the same diagnostic value. Therefore, one can avoid to measure more than one of these peptides and/or proteins, since measuring additional members of the same surrogate network does not give any additional diagnostic information. In addition, it is possible to choose those peptides and/or proteins of a surrogate network, which can be measured with the least effort. Both strategies can be combined to save time, resources, valuable or limited patient samples, etc. For determining these surrogate networks and peptide and/or protein panels, methods such as the methods described in PCT/EP 2005/000090 can be used.

TABLE 1
Changes of mass spectrometric signals of peptides or proteins depending on inhibitors
(AB and/or Contr.In.)

| | | | | Correlation of mass spectrometric signal with | | | | | | |
|---|---|---|---|---|---|---|---|---|---|---|
| | | | | concentration | | | | | | |
| | | | | of inhibitor | | | DPP4 inhibition by | | | |
| Peptide/ | | | | AB & | | Contr. | AB & | | Contr. | |
| Protein | | | | Contr.In. | AB | In. | Contr.In. | AB | In. | |
| No. | Frac. | m/z | Signal | A | B | C | D | E | F | Sequence |
| 1 | 23 | 1737 | 647 | — | — | — | — | — | pos. | Col a1(I) rat 425-443 |
| 2 | 42 | 2323 | 610 | pos. | — | — | — | — | — | Col a1(I) rat 469-493 |
| 3 | 30 | 1369 | 392 | — | — | pos. | — | — | — | Col a1(II) rat 1168-1182 |
| 4 | 30 | 1446 | 273 | — | — | pos. | — | — | — | Col a2(I) rat 1097-1113 |
| 5 | 49 | 2607 | 237 | — | — | — | — | pos. | — | FibA rat 333-358 |
| 6 | 30 | 3335 | 176 | — | — | pos. | — | — | — | |
| 7 | 31 | 3318 | 136 | — | — | pos. | — | — | — | |
| 8 | 52 | 1363 | 89 | — | pos. | — | — | — | — | |
| 9 | 58 | 2675 | 62 | — | — | — | pos. | — | — | |
| 10 | 30 | 3351 | 59 | — | — | pos. | — | — | — | *Col a1(III) mouse |
| | | | | | | | | | | 275-310 |
| 11 | 40 | 2244 | 58 | — | pos. | — | — | pos. | — | |
| 12 | 25 | 1849 | 55 | — | — | — | — | — | pos. | |
| 13 | 42 | 3187 | 54 | — | pos. | — | — | pos. | — | |
| 14 | 25 | 1721 | 49 | — | — | pos. | — | — | pos. | |
| 15 | 30 | 3469 | 43 | — | — | pos. | — | — | — | |
| **16 | 51 | 2676 | 43 | — | — | pos. | — | — | pos. | |
| 17 | 56 | 2744 | 38 | — | pos. | — | — | — | — | |
| 18 | 56 | 5489 | 38 | — | pos. | — | — | pos. | — | |
| 19 | 25 | 1436 | 36 | — | — | — | pos. | — | — | |
| 20 | 60 | 2751 | 23 | — | pos. | — | — | pos. | — | |
| 21 | 28 | 2940 | 544 | — | — | neg. | — | — | — | |
| 22 | 49 | 2336 | 470 | — | — | neg. | — | — | neg. | |
| 23 | 26 | 3267 | 458 | — | — | neg. | — | — | neg. | |
| 24 | 79 | 2711 | 452 | — | — | — | neg. | — | — | |
| 25 | 25 | 3267 | 409 | — | — | — | — | — | neg. | |
| 26 | 73 | 3868 | 364 | — | — | — | — | neg. | — | |
| 27 | 25 | 3210 | 297 | — | — | — | — | — | neg. | |
| 28 | 25 | 2094 | 281 | — | — | — | neg. | — | — | |
| 29 | 30 | 2589 | 261 | — | — | — | neg. | — | — | |
| 30 | 74 | 3867 | 241 | — | — | — | — | neg. | — | |
| 31 | 31 | 3148 | 234 | neg. | — | — | neg. | — | — | |
| 32 | 39 | 2039 | 193 | — | — | neg. | — | — | neg. | |
| 33 | 38 | 4086 | 191 | — | — | — | — | — | neg. | |
| 34 | 76 | 2711 | 182 | — | — | — | neg. | — | — | |
| 35 | 29 | 2603 | 174 | — | — | neg. | — | — | neg. | |
| 36 | 28 | 3680 | 172 | — | — | neg. | — | — | — | |
| 37 | 27 | 3251 | 165 | — | — | neg. | neg. | — | — | |
| 38 | 77 | 2711 | 164 | — | — | — | — | neg. | — | |
| 39 | 68 | 1562 | 161 | — | neg. | — | — | — | — | |
| 40 | 59 | 2475 | 146 | — | — | neg. | — | — | — | |
| 41 | 23 | 2650 | 142 | — | — | neg. | — | — | — | |
| 42 | 68 | 3083 | 136 | — | — | — | — | neg. | — | |
| 43 | 30 | 2532 | 132 | — | — | — | — | — | neg. | |
| 44 | 37 | 1679 | 119 | neg. | — | — | neg. | — | — | |
| 45 | 21 | 2579 | 112 | — | — | — | — | — | neg. | Protocol III a1 rat |
| | | | | | | | | | | 812-839 |
| 46 | 75 | 2711 | 104 | — | — | — | — | neg. | — | |
| 47 | 23 | 1938 | 101 | — | — | neg. | — | — | — | |
| 48 | 26 | 1633 | 100 | — | — | neg. | — | — | neg. | |
| 49 | 28 | 2547 | 92 | — | — | neg. | — | — | neg. | Col 1a rat 805-832 |
| 50 | 25 | 3283 | 92 | — | — | — | — | — | neg. | |
| 51 | 26 | 3210 | 92 | — | — | neg. | — | — | — | |
| 52 | 73 | 1934 | 91 | — | neg. | — | — | neg. | — | |
| 53 | 25 | 2454 | 87 | — | — | neg. | — | — | neg. | |
| 54 | 38 | 3493 | 87 | — | — | neg. | — | — | — | |
| 55 | 25 | 3358 | 85 | — | — | neg. | — | — | — | |
| **56 | 39 | 2097 | 82 | — | — | neg. | — | — | neg. | |
| 57 | 38 | 2041 | 80 | — | — | neg. | — | — | — | |
| 58 | 51 | 2654 | 75 | — | — | neg. | — | — | — | |
| 59 | 28 | 3696 | 75 | — | — | neg. | — | — | — | |
| 60 | 30 | 1583 | 74 | — | — | — | neg. | — | — | |
| 61 | 26 | 2501 | 70 | — | — | neg. | — | — | — | |
| 62 | 24 | 3000 | 70 | — | — | neg. | — | — | neg. | |
| 63 | 36 | 3410 | 67 | — | — | neg. | — | — | — | |
| 64 | 27 | 3194 | 64 | — | — | neg. | — | — | — | |
| 65 | 33 | 2938 | 63 | — | — | neg. | — | — | neg. | |
| 66 | 95 | 1634 | 61 | — | — | — | — | neg. | — | |
| 67 | 77 | 5390 | 56 | — | — | — | — | neg. | — | |
| 68 | 27 | 2337 | 56 | — | — | neg. | — | — | neg. | |
| 69 | 15 | 1510 | 55 | — | — | neg. | — | — | neg. | |
| 70 | 35 | 2049 | 55 | — | — | neg. | — | — | — | |
| 71 | 26 | 2234 | 54 | — | — | neg. | — | — | neg. | |
| 72 | 28 | 4922 | 54 | — | — | neg. | — | — | neg. | |
| 73 | 28 | 2192 | 52 | — | — | neg. | — | — | — | |
| 74 | 28 | 2220 | 52 | neg. | — | — | — | — | — | |
| 75 | 20 | 1277 | 50 | — | — | — | — | — | neg. | |
| 76 | 28 | 2563 | 49 | — | — | neg. | — | — | neg. | |
| 77 | 22 | 2283 | 49 | — | — | neg. | — | — | neg. | |
| 78 | 33 | 2239 | 49 | — | — | neg. | — | — | — | |
| 79 | 67 | 2473 | 48 | — | — | — | — | neg. | — | |
| 80 | 56 | 4359 | 47 | — | — | — | — | neg. | — | Col a2(I) rat 545-590 |
| 81 | 32 | 3132 | 46 | — | — | — | — | — | neg. | |
| 82 | 32 | 2829 | 46 | — | — | neg. | — | — | — | |
| 83 | 35 | 4228 | 42 | neg. | — | — | — | — | — | |
| 84 | 68 | 3120 | 42 | — | neg. | — | — | neg. | — | |
| 85 | 25 | 3226 | 42 | — | — | — | — | — | neg. | |
| 86 | 16 | 1510 | 41 | — | — | neg. | — | — | neg. | |
| 87 | 35 | 4720 | 41 | — | — | — | — | — | neg. | |
| 88 | 27 | 3359 | 40 | — | — | — | neg. | — | — | |
| 89 | 38 | 4286 | 40 | — | — | neg. | neg. | — | — | |
| 90 | 27 | 3266 | 39 | — | — | neg. | — | — | neg. | |
| 91 | 58 | 2474 | 37 | — | — | — | neg. | — | — | |
| 92 | 23 | 2855 | 37 | — | — | neg. | — | — | neg. | |
| 93 | 29 | 2220 | 37 | — | — | — | — | — | neg. | |
| 94 | 34 | 5876 | 36 | — | — | — | neg. | — | — | |
| 95 | 36 | 4719 | 36 | — | — | neg. | neg. | — | — | |
| 96 | 28 | 2579 | 35 | — | — | neg. | — | — | — | |
| 97 | 27 | 1625 | 32 | — | — | neg. | — | — | — | |
| 98 | 31 | 2814 | 30 | — | — | — | — | — | neg. | |
| 99 | 36 | 5844 | 30 | — | — | neg. | neg. | — | — | |
| 100 | 31 | 4467 | 28 | — | — | — | — | — | neg. | |
| 101 | 24 | 2632 | 27 | — | — | — | — | — | neg. | |
| 102 | 26 | 3283 | 24 | — | — | — | — | — | neg. | |
| 103 | 35 | 4356 | 24 | neg. | — | — | — | — | — | |
| 104 | 35 | 4735 | 24 | neg. | — | — | — | — | — | |
| 105 | 36 | 2332 | 19 | — | — | neg. | — | — | — | |
| 106 | 52 | 2638 | 17 | — | — | neg. | — | — | neg. | |

*The peptide sequence determined presumably represents a murine sequence (according to sequence database searches performed). However, as the sample used to identify this sequence was from rat, the peptide sequence determined represents a rat sequence not present in the databases. This sequence seems to be conserved between mice and rats.
**Peptide/Protein 56 and 16 are combined to panel 62 (Table 2, FIG. 5), to improve the correlation coefficient.
Frac. = Fraction;
m/z = mass/charge;
Signal = mass spectrometric signal intensity in arbitrary units [au];
AB = AB192 DPP4 inhibitor;
Contr.In. = control inhibitor;
A to F relate to the different experimental groups as described above. With positive (pos.) correlation regarding the inhibitor concentration is meant that higher concentrations of inhibitor result in higher concentration of a peptide or protein.

With positive correlation regarding the before/after change in DPP4 inhibition is meant, that less DPP4 activity results in lower concentration of a peptide or protein. Peptide or protein numbers 1 to 20 of Table 1 are increased in their amount present in plasma of DPP4 inhibitor treated rats and supposedly represent substrates of DPP4 and/or DPP8, peptide or protein numbers 21 to 106 of Table 1 are decreased in their amount present in plasma of inhibitor-treated rats and supposedly represent products of DPP4 and/or DPP8. Peptides are sorted firstly by positively correlating (peptides 1-20) and negative correlating (peptides 21-106) peptides and secondly by their respective mass spectrometric signal intensity (row “Signal”).

The sequences of peptides no. 2, 3 and 45 of Table 1 were determined and are as follows:

peptide no. 2:

(SEQ ID NO:1)

LPGPPGERGGPGSRGFPGADGVAGP

fragment of collagen alpha 1 (amino acid positions 469-493; Database: UniProtKB_Q63079)

4 out of 6 proline residues are hydroxy-proline residues

peptide no. 3:

(SEQ ID NO:2)

PPGPPGPPGPPGPPS

Fragment of collagen alpha 1 (amino acid positions 1168-1182; UniProtKB_Q63079)

4 out of 10 proline residues are hydroxy-proline residues

peptide no. 45:

(SEQ ID NO:3)

GAPGQNGEPGAKGERGAPGEKGEGGPPG

fragment of procollagen, type III, alpha I (amino acid positions 812-839; UniProtKB_Q5PQT6)
4 out of 5 proline residues are hydroxy-proline residues

The analysis of the sequenced peptides 2, 3 and 45 revealed that these peptides possess the consensus sequence for DPP4 cleavage, which is Xxx-Pro/Ala-Yyy, wherein Xxx is any amino acid residue and Yyy is any amino acid residue except Prolin and except a hydroxylated amino acid. DPP4 cleaves this consensus sequence between Pro/Ala and Yyy, resulting in a peptide having the two amino acids Xxx-Pro/Ala at the C-terminus and a peptide having the amino acid Yyy at the N-terminus. Therefore, all three identified peptides no. 2, 3 and 45 are potential substrates of DPP4, potentially accumulating in vivo in rats treated with a DPP4 inhibitor. Thus, in a preferred embodiment of the method of the invention, the at least one protease is DPP4 and the peptides and/or proteins to be determined comprise one or more peptides selected from the group consisting of peptides no. 2, 3 and 45.

TABLE 2
Panels of pairs of peptides or proteins of Table 1 improving the
overall correlation to inhibitor concentration (AB and/or Contr.In.) or to
inhibitor-mediated decreased DPP4 activity

| | Peptide/ | | Peptide/ | | Correlation coefficient of | | |
|---|---|---|---|---|---|---|---|
| Panel | Protein 1 | | Protein 2 | | | Pep./ | Panel 1 + |
| No. | Frac. | m/z | Frac. | m/z | Pep./Prot. 1 | Prot. 2 | 2 |
| | | | | | with concentration | | |
| | | | | | of AB and Contr.In. | | |
| 1 | 37 | 1679 | 35 | 4228 | −0.35 | −0.38 | 0.43 |
| 2 | 31 | 3148 | 35 | 4356 | −0.52 | −0.52 | 0.59 |
| | | | | | with concentration of AB | | |
| 3 | 73 | 1934 | 56 | 2744 | −0.44 | 0.48 | 0.66 |
| 4 | 68 | 3120 | 56 | 2744 | −0.48 | 0.48 | 0.61 |
| | | | | | with concentration of Contr.In. | | |
| 5 | 31 | 3318 | 51 | 2676 | 0.44 | 0.38 | 0.66 |
| 6 | 30 | 3335 | 51 | 2676 | 0.49 | 0.38 | 0.69 |
| 7 | 31 | 3318 | 27 | 3266 | 0.44 | −0.47 | 0.65 |
| 8 | 28 | 2940 | 51 | 2676 | −0.42 | 0.38 | 0.63 |
| 9 | 28 | 3696 | 51 | 2676 | −0.45 | 0.38 | 0.64 |
| 10 | 28 | 3680 | 51 | 2676 | −0.45 | 0.38 | 0.64 |
| 11 | 32 | 2829 | 51 | 2676 | −0.45 | 0.38 | 0.63 |
| 12 | 25 | 2454 | 51 | 2676 | −0.38 | 0.38 | 0.56 |
| 13 | 31 | 3318 | 33 | 2239 | 0.44 | −0.5 | 0.61 |
| 14 | 30 | 3335 | 27 | 3266 | 0.49 | −0.47 | 0.65 |
| 15 | 28 | 4922 | 30 | 3469 | −0.59 | 0.6 | 0.72 |
| 16 | 30 | 1369 | 30 | 3335 | 0.52 | 0.49 | 0.66 |
| 17 | 30 | 3335 | 33 | 2239 | 0.49 | −0.5 | 0.63 |
| 18 | 30 | 3351 | 51 | 2676 | 0.6 | 0.38 | 0.73 |
| 19 | 31 | 3318 | 27 | 1625 | 0.44 | −0.42 | 0.57 |
| 20 | 31 | 3318 | 25 | 3358 | 0.44 | −0.46 | 0.57 |
| 21 | 31 | 3318 | 27 | 3194 | 0.44 | −0.45 | 0.57 |
| 22 | 59 | 2475 | 51 | 2676 | −0.52 | 0.38 | 0.64 |
| 23 | 30 | 3335 | 36 | 2332 | 0.49 | −0.5 | 0.61 |
| 24 | 31 | 3318 | 32 | 2829 | 0.44 | −0.45 | 0.56 |
| 25 | 30 | 3351 | 30 | 3469 | 0.6 | 0.6 | 0.71 |
| 26 | 30 | 3351 | 28 | 4922 | 0.6 | −0.59 | 0.71 |
| 27 | 33 | 2938 | 30 | 3351 | −0.6 | 0.6 | 0.71 |
| 28 | 51 | 2654 | 51 | 2676 | −0.56 | 0.38 | 0.67 |
| 29 | 31 | 3318 | 23 | 1938 | 0.44 | −0.41 | 0.55 |
| 30 | 31 | 3318 | 27 | 2337 | 0.44 | −0.43 | 0.55 |
| 31 | 36 | 3410 | 51 | 2676 | −0.39 | 0.38 | 0.51 |
| 32 | 27 | 3251 | 16 | 1510 | −0.55 | −0.48 | 0.65 |
| 33 | 30 | 3335 | 16 | 1510 | 0.49 | −0.48 | 0.59 |
| 34 | 30 | 3335 | 27 | 1625 | 0.49 | −0.42 | 0.59 |
| 35 | 30 | 3335 | 32 | 2829 | 0.49 | −0.45 | 0.59 |
| 36 | 28 | 3680 | 31 | 3318 | −0.45 | 0.44 | 0.56 |
| 37 | 31 | 3318 | 25 | 2454 | 0.44 | −0.38 | 0.54 |
| | | | | | with DPP4 activity in | | |
| | | | | | presence of AB and Contr.In. | | |
| 38 | 27 | 3251 | 27 | 3359 | −0.28 | −0.44 | 0.55 |
| | | | | | with DPP4 activity in presence of AB | | |
| 39 | 68 | 3083 | 60 | 2751 | −0.38 | 0.77 | 0.84 |
| 40 | 95 | 1634 | 60 | 2751 | −0.4 | 0.77 | 0.84 |
| 41 | 56 | 4359 | 56 | 5489 | −0.4 | 0.67 | 0.81 |
| 42 | 95 | 1634 | 68 | 3120 | −0.4 | −0.45 | 0.6 |
| 43 | 56 | 4359 | 68 | 3120 | −0.4 | −0.45 | 0.59 |
| 44 | 77 | 2711 | 95 | 1634 | −0.38 | −0.4 | 0.54 |
| 45 | 73 | 1934 | 56 | 4359 | −0.38 | −0.4 | 0.54 |
| 46 | 73 | 1934 | 95 | 1634 | −0.38 | −0.4 | 0.52 |
| 47 | 42 | 3187 | 60 | 2751 | 0.74 | 0.77 | 0.87 |
| 48 | 74 | 3867 | 56 | 4359 | −0.45 | −0.4 | 0.57 |
| 49 | 73 | 3868 | 56 | 4359 | −0.42 | −0.4 | 0.54 |
| 50 | 75 | 2711 | 67 | 2473 | −0.38 | −0.39 | 0.49 |
| 51 | 68 | 3083 | 75 | 2711 | −0.38 | −0.38 | 0.49 |
| | | | | | with DPP4 | | |
| | | | | | activity in presence of Contr.In. | | |
| 52 | 25 | 2454 | 51 | 2676 | −0.4 | 0.55 | 0.69 |
| 53 | 21 | 2579 | 51 | 2676 | −0.43 | 0.55 | 0.71 |
| 54 | 26 | 1633 | 51 | 2676 | −0.43 | 0.55 | 0.69 |
| 55 | 28 | 4922 | 51 | 2676 | −0.4 | 0.55 | 0.64 |
| 56 | 25 | 3267 | 39 | 2039 | −0.47 | −0.59 | 0.68 |
| 57 | 26 | 3267 | 51 | 2676 | −0.46 | 0.55 | 0.66 |
| 58 | 26 | 2234 | 51 | 2676 | −0.48 | 0.55 | 0.65 |
| 59 | 25 | 3267 | 16 | 1510 | −0.47 | −0.51 | 0.63 |
| 60 | 25 | 3267 | 21 | 2579 | −0.47 | −0.43 | 0.63 |
| 61 | 39 | 2039 | 38 | 4086 | −0.59 | −0.41 | 0.73 |
| *62 | 39 | 2097 | 51 | 2676 | −0.58 | 0.55 | 0.73 |
| 63 | 51 | 2676 | 24 | 2632 | 0.55 | −0.46 | 0.7 |
| 64 | 15 | 1510 | 51 | 2676 | −0.51 | 0.55 | 0.66 |
| 65 | 16 | 1510 | 31 | 4467 | −0.51 | −0.54 | 0.65 |
| 66 | 39 | 2039 | 51 | 2676 | −0.59 | 0.55 | 0.72 |
| 67 | 30 | 2532 | 51 | 2676 | −0.55 | 0.55 | 0.69 |
| 68 | 49 | 2336 | 51 | 2676 | −0.52 | 0.55 | 0.65 |
| 69 | 25 | 1721 | 51 | 2676 | 0.51 | 0.55 | 0.65 |
| 70 | 32 | 3132 | 51 | 2676 | −0.55 | 0.55 | 0.68 |
| 71 | 51 | 2676 | 27 | 3266 | 0.55 | −0.49 | 0.67 |
| 72 | 29 | 2603 | 51 | 2676 | −0.53 | 0.55 | 0.66 |
| 73 | 16 | 1510 | 52 | 2638 | −0.51 | −0.49 | 0.64 |
| 74 | 51 | 2676 | 26 | 3283 | 0.55 | −0.48 | 0.67 |
| 75 | 25 | 3283 | 16 | 1510 | −0.54 | −0.51 | 0.66 |
| 76 | 25 | 3267 | 26 | 1633 | −0.47 | −0.43 | 0.59 |
| 77 | 51 | 2676 | 29 | 2220 | 0.55 | −0.53 | 0.66 |
| 78 | 39 | 2039 | 28 | 2547 | −0.59 | −0.48 | 0.68 |

Frac. = Fraction;
m/z = mass/charge;
AB = AB192 inhibitor;
Contr.In. = control inhibitor;
Pep./Prot. = Peptide/Protein = peptide or protein;
Panel 1 + 2 = panel made by use of Peptide/Protein 1 and Peptide/Protein 2.
*This peptide/protein panel is shown in FIG. 5 and results in an improvement of the correlation coefficient for Peptide/Protein no. 56 and 16 from Table 1 from r = −0.58 and r = 0.55 for the individual peptides/proteins, respectively, to r = 0.73 for the peptide panel combining both peptides/proteins.

### Example 8

### In Vivo Inhibition of Factor Xa

Male sprague Dawley-rats (350-400 g) were used for the in vivo studies. The animals were anaesthetized using Ketamin/Xylazin (13 mg/kg and 1 mg/kg i.p.) followed by a continuous infusion of 4.5 and 0.5 mg/kg/h, respectively, during the experiment. Animals were kept at 38° C.

Rats received a bolus injection of 3 μmol/kg i.v. and a continuous infusion of 1.98 μmol/kglh of a clotting inhibitor over a period of 2 hours via the right V. jugularis. The inhibitor used was a Factor Xa inhibitor. 1 h after start of the continuous infusion of the clotting inhibitor, a clotting activator was infused for 1 h (7.5 ml/kg/h; prediluted 1:10 in 0.9% NaCl) via the left V. jugularis. The clotting activator used contained thromboplastin (tissue factor) which converts the protein prothrombin into thrombin in a complex series of reactions thereby also activating Factor X resulting in generation of the active protease Factor Xa. Blood samples were collected in citrate containers (0.13 molyl) via the left A. carotis before infusion of the clotting inhibitor, 30 min after the start of the infusion of the clotting activator, and at the end of the infusion of the clotting activator (clotting inhibitor group).

One control group of rats (clotting activator control) received 0.9% NaCl solution instead of clotting inhibitor, but was otherwise treated in the same way as the rats treated with clotting inhibitor and clotting activator.

A second control group of rats (vehicle control) received only a 0.9% NaCl solution instead of clotting inhibitor and clotting activator, but was otherwise treated in the same way as the other two groups.

All blood samples were immediately centrifuged at 4° C. for 15 min at 1500×g and stored at −20° C. until determination of thrombin-antithrombin (TAT) complexes. The TAT-complexes were determined using standard clinical methods known in the art (e.g. those described in Example 9).

Blood samples (6 ml) for determination of peptide/protein compositions (prepared according to Examples 3 to 6) were collected at the end of the experiment (120 min) via the right A. carotis in EDTA (2.5 mg/ml) containers and immediately centrifuged (room temperature, 2000×g, 10 min). The supernatant was taken and additionally centrifuged for 15 min at room temperature and 2500×g to prepare platelet poor plasma. Samples were immediately stored at −80° C.

### Example 9

### Determination of Clotting Activity

To confirm that the clotting activator in vivo activated the clotting cascade in the rats, and to confirm that the clotting inhibitor in vivo inhibited the clotting activator-mediated activation of the clotting cascade, an ELISA was done detecting TAT-complexes. For this purpose, a commercially available TAT-complex ELISA (Dade Behring Holding GmbH, Liederbach, Germany) was used according to the manufacturer's instructions.

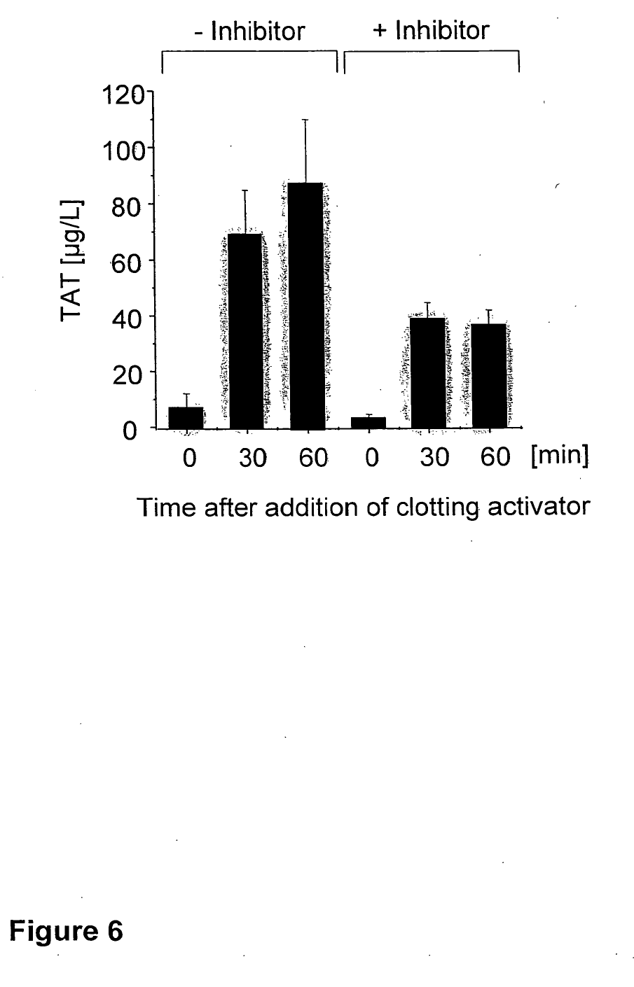

As sample rat plasma (prepared according to Example 8) was used. The results of the TAT-complex ELISA are shown in FIG. 6.

### Example 10

### Comparison of Peptide/Protein Patterns

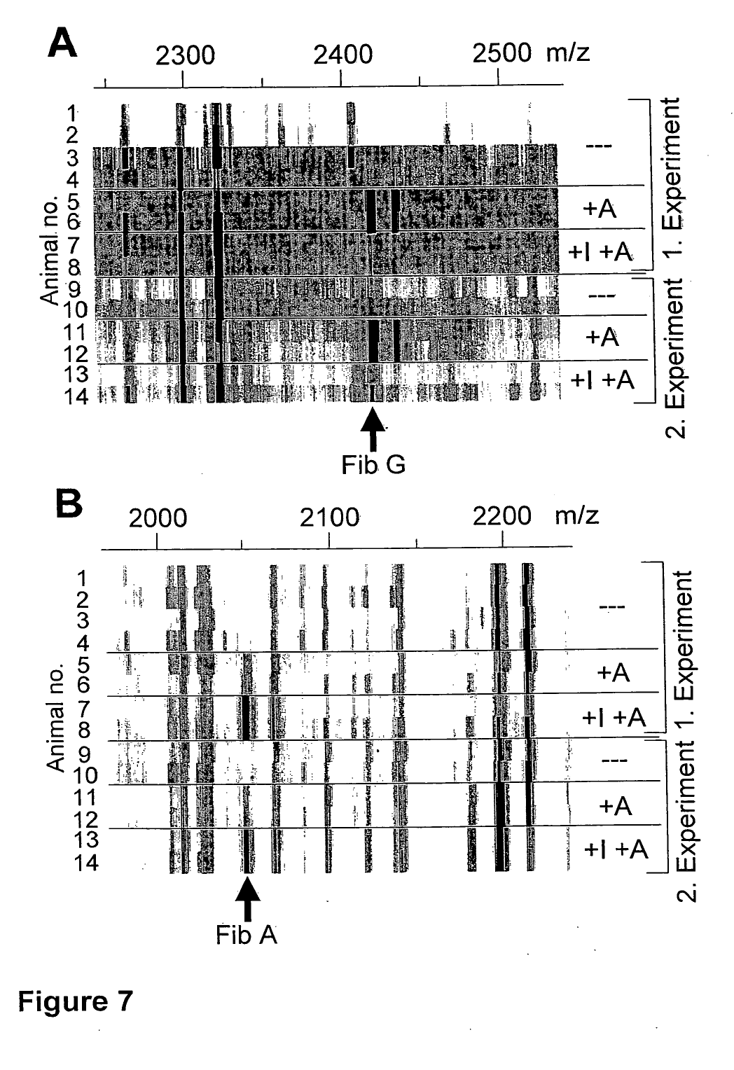

In order to demonstrate that the peptide/protein pattern of a biological sample may be altered by administration of a compound or composition to a living organism, rats were infused either 0.9% saline (vehicle control ( - - - ) group), 0.9% saline together with a clotting activator (clotting activator control (+A) group) or clotting inhibitor and subsequently clotting activator (clotting inhibitor (+I+A) group). As shown in FIG. 7A, the arrow indicates a peptide, which is present in rat plasma only if clotting has been activated (the peptide is missing in vehicle control ( - - - ) group and present in +A group), but which is clearly reduced if the clotting inhibitor is simultaneously present (+I+A group). Therefore, this peptide most likely is generated due to the clotting cascade and its generation is inhibited by the clotting inhibitor. Thus, this peptide most likely represents a marker for the in vivo activity of the clotting inhibitor used.

The identity of the peptide was determined as a fragment of Fibrinogen G (Fib G), representing amino acids 89 to 10. In FIG. 7B, the arrow indicates a peptide which is more dominantly present in rat plasma if the protease inhibitor is present. In the absence of clotting activator and clotting inhibitor (vehicle control ( - - - ) group) and in the presence of the clotting activator alone (+A group), this peptide is absent or present at low levels only. Consequently, this peptide may be generated due to an unexpected protease activity caused by the presence of the clotting inhibitor. These kinds of reactions among others potentially could indicate side effects of a drug containing this clotting inhibitor.

Alternatively this peptide could indicate that the clotting inhibitor does not inhibit all proteolytic processes associated with clotting. The peptide of panel B is a fragment of Fibrinogen A (Fib A) representing amino acids 520 to 538 of Fibrinogen A. Both of the peptides Fib G and Fib A potentially could also be used to distinguish the clotting inhibitor used in this example, which is specific for factor Xa, from other clotting inhibitors.

The sequence of the fragment of Fib A was determined and is as follows: MADEAASEAHQEGDTRTTK (SEQ ID NO:4). Fib A contains plasmin cleavage sites between amino acid positions 519 and 520, and amino acid positions 538 and 539, respectively. Therefore, it is expected that said fragment of Fib A having the amino acid sequence according to SEQ ID NO:4 is generated due to the activity of plasmin. Thus, in a preferred embodiment of the method of the invention, the at least one protease is factor Xa and the peptides and/or proteins to be determined comprise the peptide having the amino acid sequence according to SEQ ID NO:4.

### Example 1

### Comparison of Human Serum and Human Plasma

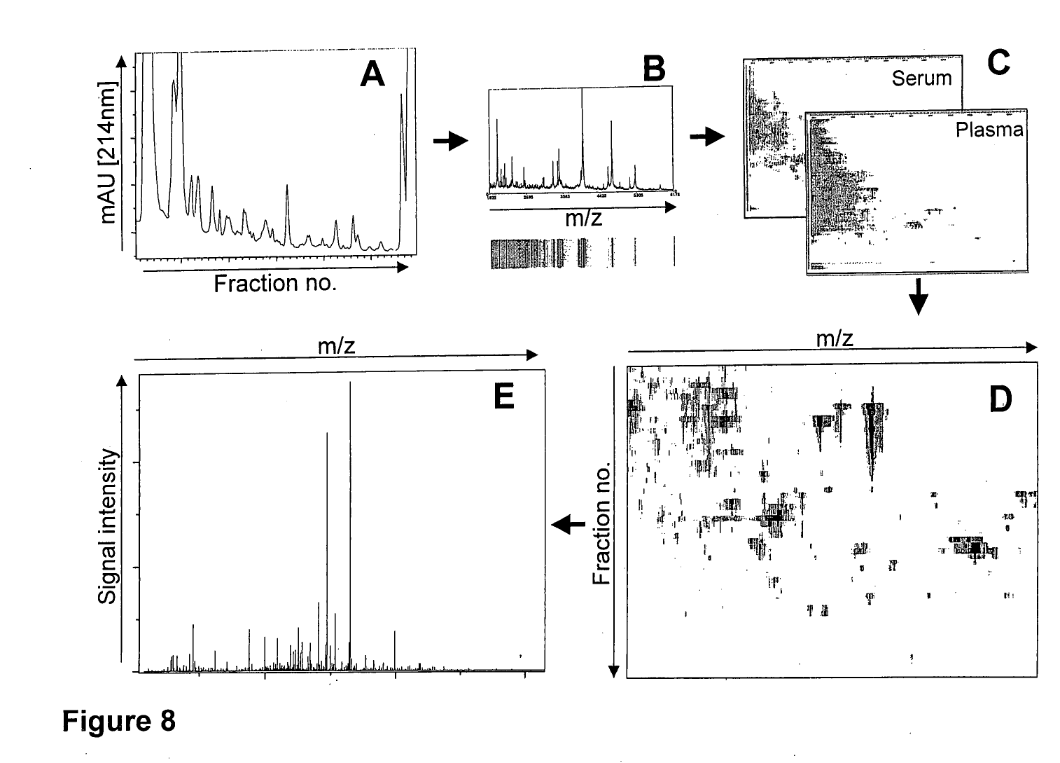

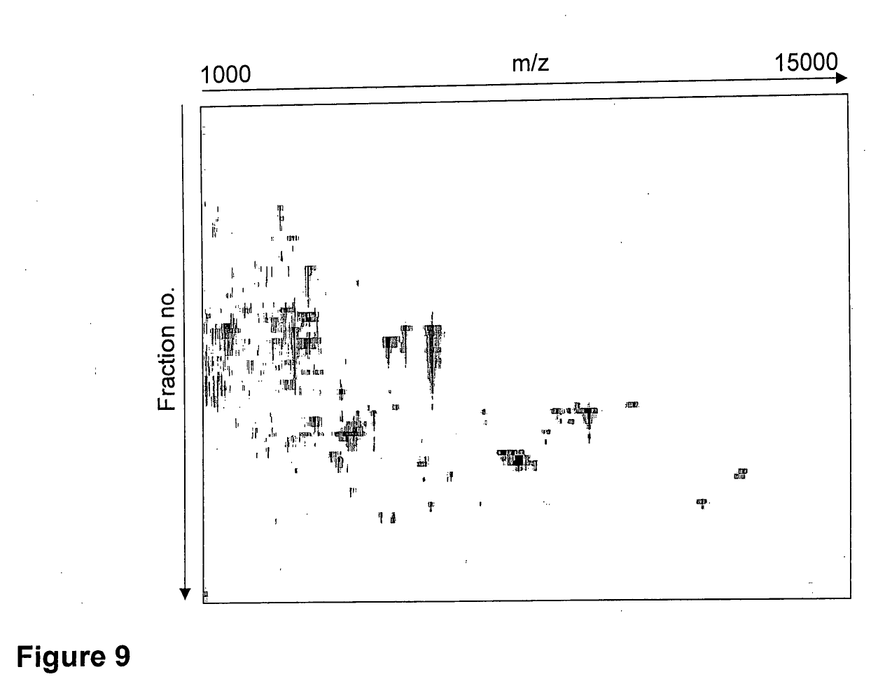

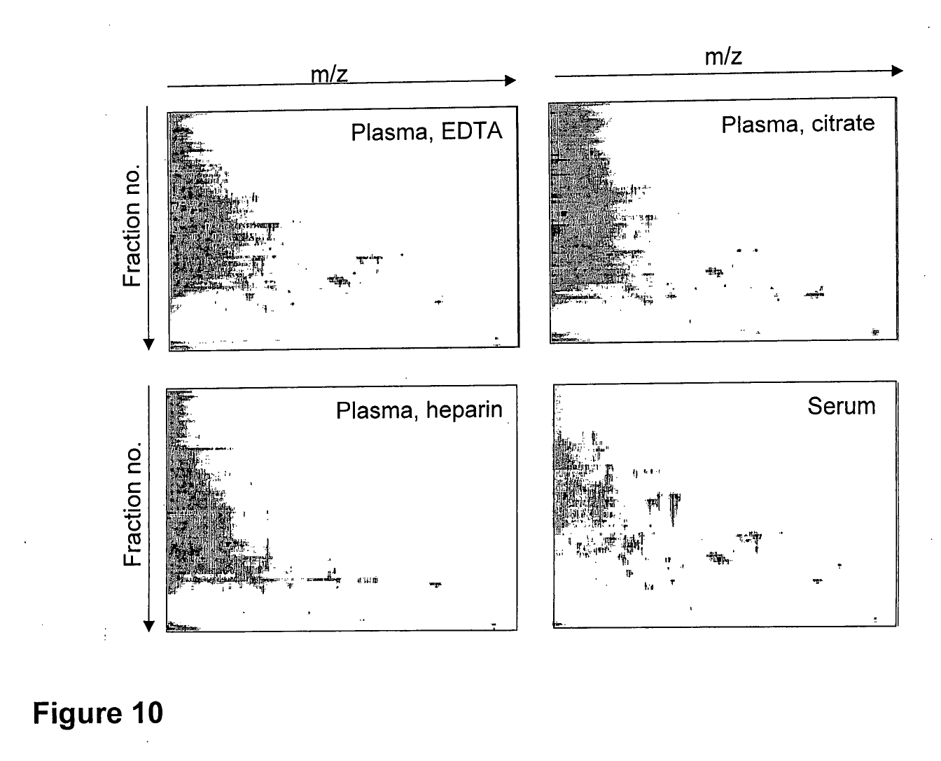

Human serum and plasma was prepared from healthy subjects according to standard methods known in the art. The majority of peptides and/or proteins present in serum/plasma was removed by precipitation using trichloracetic acid resulting in enrichment of small peptides ≦15 kDa. Subsequently, a reversed phase chromatography resulting in 96 fractions from each sample was done as described in Example 4. An exemplary reversed phase chromatogram is shown in FIG. 8A. Subsequently each individual fraction was analyzed by mass spectrometry as described in Example 5. Each mass spectrum was converted into a “gel-like” view by converting the peak hight into color (grey scale) intensity (FIG. 8B). 96 “gel-like” views of the mass spectra of each fraction of one sample were combined to form a so called “peptide display” of that sample (FIG. 8C). These peptide displays contain about 3000 to 7000 mass spectrometric signals, which correspond to 1500 to 3000 individual peptides and/or proteins. This is due to the fact, that identical peptides and/or proteins may elute in adjacent fractions or may be detected in the mass spectrometer in differently charged states. For example, the same peptide could be present in a peptide display as a single charged peptide of mass 500 and as a second signal of a double charged peptide of a “mass” of 250. Peptide displays were generated from serum as well as plasma. Peptide displays were generated from plasma prepared using different anticoagulants such as citrate, EDTA or heparin. All of these substances inhibit the coagulation cascade by inhibiting the activation of a series of proteases involved in the coagulation cascade. As a consequence, many of the peptides and/or proteins generated by the activity of the proteases of the coagulation cascade are not present in plasma, whereas these peptides and/or proteins generated by the activity of the proteases of the coagulation cascade are present in serum (cf. e.g. FIG. 10). The peptide displays of serum and plasma were electronically subtracted from each other, resulting in a so called “differential peptide display” depicting the differences in the peptide and/or protein composition of serum as compared to plasma (FIG. 8D and FIG. 9). Individual peptides and/or proteins of the “differential peptide display” can be sequenced by use of methods known to the skilled person, e.g. those described in Example 6. An exemplary mass spectrum of such a peptide is shown in FIG. 8E. A high number of peptides and/or proteins, many of them in rather high abundance, are only present in serum and are not detectable in plasma. These peptides and/or proteins are derived from coagulation processes, enzymatic activities or from the release of peptides and/or proteins from the blood clot during coagulation. Using methods such as those described in Example 6, we have identified fragments originating form various precursors or extracellular peptides and/or proteins, which differ in their amount present in serum and plasma, such as fragments of apolipoprotein A-1 (Apo-A1), collagen alpha(I), complement C3, complement C4, fibrinogen alpha/alpha-E chain, fibrinogen beta chain, prothrombin and serum albumin. In addition, we found fragments of several intracellular peptides and/or proteins which differ in their amount present in serum and plasma, such as myosin heavy chain, talin 1, thymosin beta-4, transgelin 2, tubulin alpha-1 chain, tubulin beta-1 chain, vasodilator-stimulated phosphoprotein and zyxin (zyxin 2). FIG. 10 shows that many peptides and/or proteins are much more abundant in serum as compared to plasma prepared by using citrate, EDTA or heparin.

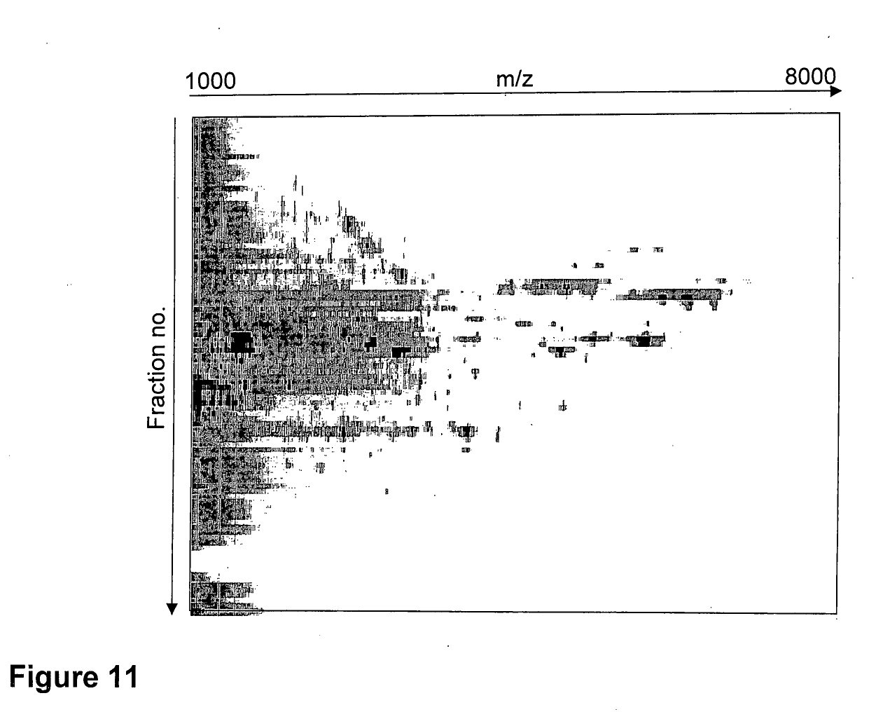

If the clotting process is allowed to take place for different periods of time the amount of peptides and/or proteins generated by the activity of the proteases of the coagulation cascade increases with time. This is shown in FIG. 11, where the peptide display of serum samples clotted for 15 min, is electronically subtracted from the peptide display of serum samples clotted for 8 hours. The time difference results in altered amounts of numerous peptides and/or proteins present in the samples and the quantitative changes reflect the time kinetics of the proteolytic activity of the proteases involved in the clotting cascade. Peptides and/or proteins the amounts of which were altered due to the increased clotting time (8 h instead of 15 min) are shown in the differential peptide display of FIG. 11. Sequence analysis of these peptides and/or proteins revealed that among others these peptides and/or proteins were fragments of fibrinogen, thymosin and zyxin.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method of determining the ability and/or the specificity of a compound or composition to modulate the activity of at least one protease comprising the steps of:
a) contacting said compound or composition with at least one protease in the presence of a plurality of peptides and/or proteins, and optionally in the presence of a protease inhibitor or protease activator, and
b) determining qualitatively and/or quantitatively the peptides and/or proteins resulting from step (a).

2. The method of claim 1, wherein step (b) is performed in a time dependent manner.

3. The method of claim 2, wherein step (b) is performed at a first time and at least a second time.

4. The method of claim 1 further comprising the step of comparing the results obtained in step (b) with a reference sample.

5. The method of claim 4, wherein said reference sample contains a known modulator of the activity of said at least one protease.

6. The method of claim 1, further comprising the step of:
c) comparing the results obtained in step (b) with the results obtained from (i) determining qualitatively and/or quantitatively the peptides and/or proteins prior to step (a) and/or (ii) determining qualitatively and/or quantitatively the peptides and/or proteins in a corresponding sample which does not contain said compound or composition and/or (iii) determining qualitatively and/or quantitatively the peptides and/or proteins in a corresponding sample which contains a different concentration of said compound or composition and/or (iv) determining qualitatively and/or quantitatively the peptides and/or proteins in a corresponding sample which contains a different compound or composition and/or (v) determining qualitatively and/or quantitatively the peptides and/or proteins in a corresponding sample which has been contacted with said compound or composition for a different period of time.

7. The method of claim 1, wherein step (a) takes place within in vitro cultured cells or within the culture medium of in vitro cultured cells.

8. The method of claim 1, wherein step (a) takes place within a living organism.

9. The method of claim 1, wherein step (a) takes place within a living organism and said method further comprises the step of:
c) comparing the results obtained in step (b) with the results obtained from at least one further living organism.

10. The method of claim 9, wherein said at least one further living organism (i) has not been exposed to said compound or composition or (ii) has been exposed to a different compound or composition or (iii) has been exposed to a different concentration of said compound or composition and/or (iv) has been exposed to said compound or composition for a different period of time.

11. The method of claim 9, wherein said first living organism and said at least one further living organism have been subjected or are subjected to different environmental conditions.

12. The method of claim 9, wherein said first living organism and said at least one further living organism are of different age, sex, weight or health condition.

13. The method of claim 8, wherein said living organism and/or said at least one further living organism is afflicted with a disease.

14. The method of claim 13, wherein said disease is selected form the group consisting of hypertension, HIV/AIDS, haemophilia, thrombosis, cancer, inflammatory diseases, type II diabetes, Alzheimer's disease, osteoporosis, and hepatitis C infection (HCV).

15. The method of claim 9, wherein said first living organism and said at least one further living organism are of the same or different species.

16. The method of claim 15, wherein said living organisms of the same species have different genetic backgrounds.

17. The method of claim 8, wherein said living organism and/or said at least one further living organism is a multicellular organism.

18. The method of claim 17, wherein said multicellular organism is a mammal, preferably a human.

19. The method of claim 8, wherein said compound or composition is delivered to said living organism and/or said at least one further living organism.

20. The method of claim 8, wherein said at least one protease is delivered to said living organism.

21. The method of claim 1, further comprising the step of comparing the results obtained in step (b) with clinical or other meta data.

22. The method of claim 1, wherein said ability of a compound or composition to modulate the activity of at least one protease is the ability to decrease or increase the activity of said at least one protease.

23. The method of claim 1, wherein said at least one protease comprises at least one protease selected from the group consisting of aspartic peptidases, cysteine peptidases, glutamic peptidases, metallo peptidases, serine peptidases, threonine peptidases, amino peptidases, carboxypeptidases, endopeptidases and exopeptidases.

24. The method of claim 23, wherein said at least one protease comprises at least one protease selected from the group consisting of angiotensin converting enzyme (ACE), ACE2, renin, neutral endopeptidase (NEP) (hypertension), HIV-1 protease, HIV-2 protease, factor Xa, factor VIIa, factor XIIIa (thrombosis), interleukin-1 converting enzyme (ICE) (inflammatory diseases), dipeptidyl peptidase 4 (DPP4) (type II diabetes), alpha-, beta-, gamma-secretase, caspase (Alzheimer's disease), cathepsin K (osteoporosis), Matrix metalloproteinases (MMPs), proteosome protease (cancer) and NS3 protease (HCV).

25. The method of claim 1, wherein said step of determining qualitatively and/or quantitatively the peptides and/or proteins comprises determining the relative or absolute quantity of said peptides and/or proteins.

26. The method of claim 1, wherein said quantitative and/or qualitative determination of said peptides and/or proteins is performed using mass spectrometric methods.

27. The method of claim 1, wherein said quantitative and/or qualitative determination of said peptides and/or proteins is performed using mass spectrometric methods in combination with separation methods for said peptides and/or proteins.

28. The method of claim 27, wherein said separation methods are performed prior to, simultaneous with and/or subsequent to said mass spectrometric methods.

29. The method of claim 27, wherein said separation methods are selected from the group consisting of gel electrophoresis, liquid chromatography, gas chromatography, capillary chromatography, thin layer chromatography, mass spectrometry, precipitation techniques, liquid phase extraction techniques, filtration and other molecular size discriminating techniques.

30. The method of claim 26, wherein said mass spectrometric methods are selected from the group consisting of MALDI mass spectrometry, ESI mass spectrometry, SELDI mass spectrometry, FAB mass spectrometry and MS/MS mass spectrometry.

31. The method of claim 1, wherein said plurality of peptides and/or proteins is comprised in a biological sample.

32. The method of claim 31, wherein said biological sample is selected from the group consisting of blood, serum, plasma, urine, liquor, bronchial aspirate, sputum, tissue extracts, cell extracts, cell culture medium.

33. The method of claim 1, further comprising obtaining said compound or composition.

34. The method of claim 1, further comprising obtaining said protease.

35. The method of claim 1, further comprising obtaining said protease inhibitor or protease activator.

36. A kit comprising at least one protease and instructions for using the protease to determine the ability and/or the specificity of a compound or composition to modulate the activity of the protease in accordance with the method of claim 1.

37. The kit of claim 36, further comprising a protease inhibitor or a protease activator.

38. A kit comprising a protease inhibitor or a protease activator and instructions for using the protease inhibitor or protease activator to determine the ability and/or the specificity of a compound or composition to modulate the activity of a protease in accordance with the method of claim 1.


SEQUENCE LISTING (4 sequences)

SEQ ID NO: 1 - protein, 25 aa, Rattus norvegicus
  MISC_FEATURE: 4 out of 6 proline residues are hydroxy-proline residues
LPGPPGERGGPGSRGFPGADGVAGP

SEQ ID NO: 2 - protein, 15 aa, Rattus norvegicus
  MISC_FEATURE: 4 out of 10 proline residues are hydroxy-proline residues
PPGPPGPPGPPGPPS

SEQ ID NO: 3 - protein, 28 aa, Rattus norvegicus
  MISC_FEATURE: 4 out of 5 proline residues are hydroxy-proline residues
GAPGQNGEPGAKGERGAPGEKGEGGPPG

SEQ ID NO: 4 - protein, 19 aa, Rattus norvegicus
MADEAASEAHQEGDTRTTK